(19) United States
(12) Patent Application Publication
(10) Pub. No.: US 2018/0009904 A1
(43) Pub. Date: Jan. 11, 2018

(54) LYSOSOMAL TARGETING AND USES THEREOF

(71) Applicant: Shire Human Genetic Therapies, Inc.

(72) Inventors: Vinayaka KOTRAIAH, North Potomac, MD (US); Bohong ZHANG, Lexington, MA (US); Michael F. CONCINO, Bolton, MA (US)

(21) Appl. No.: 15/529,355

(22) PCT Filed: Nov. 20, 2015
(86) PCT No.: PCT/US15/61958

§ 371 Date: May 24, 2017

Related U.S. Application Data
(60) Provisional application No. 62/083,639, filed on Nov. 24, 2014.

(51) Int. Cl. C07K 16/40; C12N 9/26; C12N 9/24; A61K 38/00

(52) U.S. Cl. CPC C07K 16/40; C12Y 302/0105; C12Y 304/21061; C12N 9/2405; C12N 9/2408; C12Y 302/0102; C07K 2317/24; C07K 2317/21; C07K 2317/622; C07K 2317/94; C07K 2319/06; A61K 38/00; C07K 2319/70; C07K 2319/50

(57) ABSTRACT

The invention provides compositions and methods for effective lysosomal targeting mediated by PCSK9. In particular, the compositions and methods provided by the invention may be used to treat lysosomal storage diseases such as Pompe Disease and Sanfilippo Syndrome Type B, and they may be used for targeting lysosomal enzymes to the various muscles of the human body.

### CROSS-REFERENCE TO RELATED APPLICATIONS

This application claims priority to U.S. Provisional Application No. 62/083,639, filed Nov. 24, 2014, the disclosure of which is hereby incorporated in its entirety.

### BACKGROUND

More than forty lysosomal storage diseases are caused, directly or indirectly, by the absence or deficiency of one or more lysosomal enzymes.

Pompe disease is a lysosomal storage disease caused by a deficiency or dysfunction of the lysosomal hydrolase acid alpha-glucosidase (GAA), a glycogen-degrading lysosomal enzyme. Deficiency of GAA results in lysosomal glycogen accumulation in many tissues, with cardiac and skeletal muscle tissues being most seriously affected. The combined incidence of all forms of Pompe disease is estimated to be 1:40,000. It is estimated that approximately one third of patients with Pompe disease have the rapidly progressive, fatal infantile-onset form, while the majority of patients present with the more slowly progressive, juvenile or late-onset forms.

Sanfilippo syndrome, or mucopolysaccharidosis III (MPS III), on the other hand, is a rare genetic disorder characterized by the deficiency of enzymes involved in the degradation of glycosaminoglycans (GAG). Four distinct forms of MPS III, designated MPS IIIA, B, C, and D, have been identified. Each is characterized by the absence or deficiency of a different lysosomal enzyme. Mucopolysaccharidosis type IIIB (MPS IIIB; Sanfilippo B disease) is an autosomal recessive disorder that is caused by a deficiency of the enzyme alpha-N-acetylglucosaminidase (Naglu), resulting in the accumulation of heparan sulfate in lysosomes of particularly neurons and glial cells in the brain, with additional lysosomal accumulation of heparan sulfate elsewhere. MPS IIIB manifests itself primarily in the brain.

Enzyme replacement therapy (ERT) has been used to deliver enzymes for the treatment of various lysosomal storage diseases. Normally, lysosomal enzymes are synthesized in the cytosol and then traverse the endoplasmic reticulum (ER), where they are glycosylated with N-linked, high mannose type carbohydrates. In the Golgi apparatus, high mannose carbohydrates on glycoproteins are then modified by a series of glycotransferases to become mature N-glycan; one of these modifications is the addition of mannose-6-phosphate (M6P). Proteins carrying this modification are then targeted to the lysosome via binding of the M6P moiety to the cation-independent mannose-6-phosphate receptor (CI-M6PR) and cationdependant mannose-6-phoshate receptor (CD-M6PR).

Efficacy of enzyme replacement therapy is critically dependent on proper lysosomal targeting of the replacement enzyme. However, recombinantly produced Naglu protein is characterized by a dramatic lack of M6P phosphorylation, making lysosomal targeting of this enzyme and its effective use for ERT very difficult. Similarly, for some diseases, such as Pompe, enzyme replacement therapy has shown limitations, such as limited clinical benefit resulting from poor cellular uptake of recombinant enzyme in skeletal muscle and cardiac tissues of the body (Schoser et al., Neurotherapeutics 5:569-578 (2008)).

Therefore, there remains a need to develop alternative methods for lysosomal targeting to ensure effective enzyme replacement therapy.

### SUMMARY

The present invention is, in part, based on the surprising discovery that a therapeutic, for example a replacement enzyme, can be effectively delivered to lysosomes through the use of a coupling moiety that binds specifically to a proprotein convertase protein, such as PCSK9. This proprotein convertase protein, in turn, interacts with various secondary binding proteins, such as, but not limited to, amyloid precursor-like protein 2 (APLP2), Dynamin, amyloid precursor protein (APP), autosomal recessive hypercholesterolemia (ARH) protein or low density lipoprotein receptor-related protein 8 (Lrp8), thereby facilitating cellular uptake of the therapeutic and its coupling moiety. Thus, the present invention permits targeting of a therapeutic to a lysosome in a glycosylation or M6P-independent manner and can be used to deliver enzymes with low levels of glycosylation or even with complete absence of glycosylation. Accordingly, the present invention simplifies the process of manufacturing recombinant enzymes used for replacement therapy. PCSK9 is ubiquitously expressed throughout the various tissues of the body. Thus, the present invention allows enzyme replacement therapy of diseases with manifestations within and outside the nervous system. Furthermore, many of PCSK9's potential cognate transmembrane binding partners, i.e., APLP2 and Dynamin, are known to be enriched in human skeletal muscle and the kidney (The Human Protein Atlas; Uhlen et al. Nat Biotechnol. 2010 28(12):1248-50; Uhlén et al. Mol Cell Proteomics. 2005 4(12):1920-32; Pontén et al. J Pathol. 2008 216(4):387-93; Lundberg et al. Mol Syst Biol. 2010 6:450; Pontén et al. Mol Syst Biol. 2009 5:337), which enables treatment of specifically those diseases that affect the skeletal muscle system, such as Pompe disease.

In some embodiments, the coupling moiety may be an antibody or binding fragment thereof. Data suggest that binding of PCSK9 to LDL receptor (LDLR) is necessary for LDLR-cellular internalization, recycling and removal from the extracellular space. Antagonistic antibodies to PCSK9 are being developed into therapeutics for disrupting the interaction between PCSK9 and LDLR and thus lowering serum LDL-cholesterol levels.

One exemplary anti-PCSK9 antibody—J16—that disrupts the interaction between PCSK9 and LDLR has been shown to be internalized and routed to lysosomes via its binding to PCSK9 (Devay et al., 2013). J16 is a humanized version of a mouse antibody and is a human IgG2deltaA and κ chain antibody (Liang et al., 2012). Amino acid sequence and structural information for the heavy and light chains of the J16 anti-PCSK9 antibody has been published (PDB ID codes 3SQO and 2P4E) (Liang et al., 2012).

In some embodiments, the targeted therapeutic, i.e., lysosomal enzyme, and the coupling moiety, i.e., antibody, may be expressed as a fusion protein. Fusing a lysosomal enzyme to an antibody is expected to result in serum stabilization of the fusion protein. Further, binding of the antibody portion of the fusion protein to secreted PCSK9 in circulation is expected to increase the lysosomal delivery of the lysosomal enzyme as compared to cell-surface receptor based lysosomal targeting of current enzyme replacement technology. Antibodies and Fc-fusion proteins are routinely expressed in mammalian cells and purified using affinity chromatography methods. As such, the fusion protein described in this invention can be expected to be produced using standard mammalian cells such as CHO cells, for example.

Without wishing to be held to any theory, the inventors expect that a lysosomal replacement enzyme fused to an anti-PCSK9 antibody that disrupts LDLR-PCSK9 interaction will be bound by circulating PCSK9 and then routed to lysosomes.

In one aspect, the present invention provides a targeted therapeutic comprising: (i) a lysosomal enzyme; and (ii) a coupling moiety that binds specifically to a proprotein convertase protein.

In some embodiments, the proprotein convertase protein is selected from the group consisting of PC1/3; PC2; Furin; PC4; PC5/6; PACE4, PC7, SKI-1/S1P and PCSK9. In some embodiments, the proprotein convertase is PCSK9. In some embodiments, the lysosomal enzyme is selected from Table 3. In some embodiments, the lysosomal enzyme is acid alpha-glycosidase (GAA). In some embodiments, the acid alpha-glycosidase comprises an amino acid sequence at least 80%, 90% or 95% identical to SEQ ID NO:1. In some embodiments, the acid alpha-glycosidase comprises an amino acid sequence identical to SEQ ID NO:1. In some embodiments, the lysosomal enzyme is alpha-N-acetyl-glucosaminidase (Naglu). In some embodiments, the alpha-N-acetyl-glucosaminidase comprises an amino acid sequence at least 80%, 90% or 95% identical to SEQ ID NO:4. In some embodiments, the alpha-N-acetyl-glucosaminidase comprises an amino acid sequence identical to SEQ ID NO:4. In some embodiments, the coupling moiety is a peptide. In some embodiments, the coupling moiety is fused to the lysosomal enzyme creating a fusion protein. In some embodiments, the coupling moiety is fused to the N-terminus of the lysosomal enzyme. In some embodiments, the coupling moiety is fused to the C-terminus of the lysosomal enzyme. In some embodiments, the targeted therapeutic further comprises a linker joining the lysosomal enzyme and the coupling moiety. In some embodiments, the linker is a peptide linker. In some embodiments, the peptide linker comprises a sequence of three glycine residues. In some embodiments, the peptide linker comprises a cleavage site. In some embodiments, the cleavage site comprises a lysosomal protease recognition site. In some embodiments, the coupling moiety interferes with binding between the proprotein convertase protein and an LDL receptor. In some embodiments, binding between the proprotein convertase protein and the LDL receptor is reduced by at least 50%, 80%, 85%, 90% or 95%. In some embodiments, binding of the coupling moiety to PCSK9 protein alters subsequent binding between the PCSK9 protein and one or more secondary binding proteins selected from the group consisting of Amyloid Precursor-like Protein 2 (APLP2), Dynamin, Amyloid Precursor Protein (APP), Autosomal Recessive Hypercholesterolemia (ARH) protein, Low Density Lipoprotein Receptor-related Protein 8 (Lrp8) and combinations thereof. In some embodiments, binding between the PCSK9 protein and the one or more secondary binding proteins is enhanced by at least 50%, 80%, 85%, 90% or 95%, compared to binding by PCSK9 alone. In some embodiments, the coupling moiety is an antibody or antibody fragment. In some embodiments, the antibody is a monoclonal antibody. In some embodiments, the monoclonal antibody is selected from the group consisting of a human antibody, mouse antibody and a rabbit antibody. In some embodiments, the antibody is a humanized mouse antibody. In some embodiments, the antibody is a human antibody. In some embodiments, the antibody is a pH sensitive binding antibody. In some embodiments, the antibody is a IgG2delta A and κ chain antibody. In some embodiments, the antibody fragment is a single chain scFv.

In one aspect, the present invention provides a nucleic acid encoding any of the targeted therapeutics disclosed herein.

In one aspect, the present invention provides a vector comprising any of the nucleic acid sequences disclosed herein.

In one aspect, the present invention provides a host cell comprising any of the vectors disclosed herein.

In some embodiments, the host cell is selected from the group consisting of a bacterial, yeast, insect and mammalian cell. In some embodiments, the host cell is a mammalian cell. In some embodiments, the mammalian cell is a human cell. In some embodiments, the mammalian cell is a CHO cell.

In one aspect, the present invention provides a method of producing a targeted therapeutic, the method comprising steps of: a) culturing any of the host cells disclosed herein under conditions suitable for expression of the targeted therapeutic by the host cell; and b) harvesting the targeted therapeutic expressed by the host cell.

In one aspect, the present invention provides a pharmaceutical composition comprising any of the targeted therapeutics disclosed herein, and a pharmaceutical acceptable carrier.

In one aspect, the present invention provides a method of treating a lysosomal storage disease comprising administering to a subject in need of treatment any of the pharmaceutical compositions disclosed herein.

In one aspect, the present invention provides a method of delivering a targeted therapeutic to skeletal muscle, vascular smooth muscle or cardiac muscle, including administering to a subject in need of treatment any of the pharmaceutical compositions disclosed herein.

### DEFINITIONS

In order for the present invention to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Amelioration: As used herein, the term “amelioration” is meant the prevention, reduction or palliation of a state, or improvement of the state of a subject. Amelioration includes, but does not require complete recovery or complete prevention of a disease condition. In some embodiments, amelioration includes increasing levels of relevant protein or its activity that is deficient in relevant disease tissues.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. The Fc region of naturally-occurring antibodies binds to elements of the complement system, and also toreceptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, will be understood to refer to in appropriate embodiments (unless otherwise stated or clear from context) to any of the art-known or developed constructs or formats for capturing antibody structural and functional features in alternative presentation. For example, in some embodiments, the term can refer to bi- or other multi-specific (e.g., zybodies, etc) antibodies, Small Modular ImmunoPharmaceuticals (“SMIPs™”), single chain antibodies (scAbs), cameloid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.]), or other pendant group (e.g., poly-ethylene glycol, etc.).

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Cation-independent mannose-6-phosphate receptor (CI-MPR): As used herein, the term “cation-independent mannose-6-phosphate receptor (CI-MPR)” refers to a cellular receptor that binds mannose-6-phosphate (M6P) tags on acid hydrolase precursors in the Golgi apparatus that are destined for transport to the lysosome. In addition to mannose-6-phosphates, the CI-MPR also binds other proteins including IGF-II. The CI-MPR is also known as “M6P/IGF-II receptor”, “CI-MPR/IGF-II receptor”, “CD222”, “MPR300”, “IGF-II receptor” or “IGF2 Receptor.” These terms and abbreviations thereof are used interchangeably herein.

Cell culture: These terms as used herein refer to a cell population that is gown in a medium under conditions suitable to survival and/or growth of the cell population. As will be clear to those of ordinary skill in the art, these terms as used herein may refer to the combination comprising the cell population and the medium in which the population is grown.

Diluent: As used herein, the term “diluent” refers to a pharmaceutically acceptable (e.g., safe and non-toxic for administration to a human) diluting substance useful for the preparation of a reconstituted formulation. Exemplary diluents include sterile water, bacteriostatic water for injection (BWFI), a pH buffered solution (e.g. phosphate-buffered saline), sterile saline solution, Ringer's solution or dextrose solution.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

Enzyme replacement therapy (ERT): As used herein, the term “enzyme replacement therapy (ERT)” refers to any therapeutic strategy that corrects an enzyme deficiency by providing the missing enzyme. In some embodiments, the missing enzyme is provided by intrathecal administration. In some embodiments, the missing enzyme is provided by infusing into bloodstream. Once administered, enzyme is taken up by cells and transported to the lysosome, where the enzyme acts to eliminate material that has accumulated in the lysosomes due to the enzyme deficiency. Typically, for lysosomal enzyme replacement therapy to be effective, the therapeutic enzyme is delivered to lysosomes in the appropriate cells in target tissues where the storage defect is manifest.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein. In this application, the terms “expression” and “production,” and grammatical equivalent, are used inter-changeably.

Fragment: The term “fragment” as used herein refers to polypeptides and is defined as any discrete portion of a given polypeptide that is unique to or characteristic of that polypeptide. The term as used herein also refers to any discrete portion of a given polypeptide that retains at least a fraction of the activity of the full-length polypeptide. Preferably the fraction of activity retained is at least 10% of the activity of the full-length polypeptide. More preferably the fraction of activity retained is at least 20%, 30%, 40%, 50%, 60%, 70%, 80% or 90% of the activity of the full-length polypeptide. More preferably still the fraction of activity retained is at least 95%, 96%, 97%, 98% or 99% of the activity of the full-length polypeptide. Most preferably, the fraction of activity retained is 100% of the activity of the full-length polypeptide. The term as used herein also refers to any portion of a given polypeptide that includes at least an established sequence element found in the full-length polypeptide. Preferably, the sequence element spans at least 4-5, more preferably at least about 10, 15, 20, 25, 30, 35, 40, 45, 50 or more amino acids of the full-length polypeptide.

Gene: The term “gene” as used herein refers to any nucleotide sequence, DNA or RNA, at least some portion of which encodes a discrete final product, typically, but not limited to, a polypeptide, which functions in some aspect of a cellular process. The term is not meant to refer only to the coding sequence that encodes the polypeptide or other discrete final product, but may also encompass regions preceding and following the coding sequence that modulate the basal level of expression, as well as intervening sequences (“introns”) between individual coding segments (“exons”). In some embodiments, a gene may include regulatory sequences (e.g., promoters, enhancers, poly adenylation sequences, termination sequences, Kozac sequences, tata box, etc.) and/or modification sequences. In some embodiments, a gene may include references to nucleic acids that do not encode proteins but rather encode functional RNA molecules such as tRNAs, RNAi-inducing agents, etc.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Genetic control element: The term “genetic control element” as used herein refers to any sequence element that modulates the expression of a gene to which it is operably linked. Genetic control elements may function by either increasing or decreasing the expression levels and may be located before, within or after the coding sequence. Genetic control elements may act at any stage of gene expression by regulating, for example, initiation, elongation or termination of transcription, mRNA splicing, mRNA editing, mRNA stability, mRNA localization within the cell, initiation, elongation or termination of translation, or any other stage of gene expression. Genetic control elements may function individually or in combination with one another.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Linker: As used herein, the term “linker” refers to, in a fusion protein, an amino acid sequence other than that appearing at a particular position in the natural protein and is generally designed to be flexible or to interpose a structure, such as an a-helix, between two protein moieties. A linker is also referred to as a spacer.

Lysosomal enzyme: As used herein, the term “lysosomal enzyme” refers to any enzyme that is capable of reducing accumulated materials in mammalian lysosomes or that can rescue or ameliorate one or more lysosomal storage disease symptoms. Lysosomal enzymes suitable for the invention include both wild-type or modified lysosomal enzymes and can be produced using recombinant and synthetic methods or purified from nature sources. Exemplary lysosomal enzymes are listed in Table 2.

Lysosomal enzyme deficiency: As used herein, “lysosomal enzyme deficiency” refers to a group of genetic disorders that result from deficiency in at least one of the enzymes that are required to break macromolecules (e.g., enzyme substrates) down to peptides, amino acids, monosaccharides, nucleic acids and fatty acids in lysosomes. As a result, individuals suffering from lysosomal enzyme deficiencies have accumulated materials in various tissues (e.g., CNS, liver, spleen, gut, blood vessel walls and other organs).

Lysosomal Storage Disease: As used herein, the term “lysosomal storage disease” refers to any disease resulting from the deficiency of one or more lysosomal enzymes necessary for metabolizing natural macromolecules. These diseases typically result in the accumulation of un-degraded molecules in the lysosomes, resulting in increased numbers of storage granules (also termed storage vesicles). These diseases and various examples are described in more detail below.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre and post natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Peptide: As used herein, a “peptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that peptides sometimes include “non-natural” amino acids or other entities that nonetheless are capable of integrating into a polypeptide chain, optionally. As used herein, the terms “polypeptide” and “peptide” are used inter-changeably.

Protein: As used herein, the term “protein” of “therapeutic protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant protein and Recombinant polypeptide: These terms as used herein refer to a polypeptide expressed from a host cell, that has been genetically engineered to express that polypeptide. In some embodiments, a recombinant protein may be expressed in a host cell derived from an animal. In some embodiments, a recombinant protein may be expressed in a host cell derived from an insect. In some embodiments, a recombinant protein may be expressed in a host cell derived from a yeast. In some embodiments, a recombinant protein may be expressed in a host cell derived from a prokaryote. In some embodiments, a recombinant protein may be expressed in a host cell derived from an mammal. In some embodiments, a recombinant protein may be expressed in a host cell derived from a human. In some embodiments, the recombinantly expressed polypeptide may be identical or similar to a polypeptide that is normally expressed in the host cell. In some embodiments, the recombinantly expressed polypeptide may be foreign to the host cell, i.e. heterologous to peptides normally expressed in the host cell. Alternatively, in some embodiments the recombinantly expressed polypeptide can be a chimeric, in that portions of the polypeptide contain amino acid sequences that are identical or similar to polypeptides normally expressed in the host cell, while other portions are foreign to the host cell.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Target tissues: As used herein, the term “target tissues” refers to any tissue that is affected by the lysosomal storage disease to be treated or any tissue in which the deficient lysosomal enzyme is normally expressed. In some embodiments, target tissues include those tissues in which there is a detectable or abnormally high amount of enzyme substrate, for example stored in the cellular lysosomes of the tissue, in patients suffering from or susceptible to the lysosomal storage disease. In some embodiments, target tissues include those tissues that display disease-associated pathology, symptom, or feature. In some embodiments, target tissues include those tissues in which the deficient lysosomal enzyme is normally expressed at an elevated level. As used herein, a target tissue may be a brain target tissue, a spinal cord target tissue and/or a peripheral target tissue. Exemplary target tissues are described in detail below.

Treatment: As used herein, the term “treatment” (also “treat” or “treating”) refers to any administration of a therapeutic protein (e.g., lysosomal enzyme) that partially or completely alleviates, ameliorates, relieves, inhibits, delays onset of, reduces severity of and/or reduces incidence of one or more symptoms or features of a particular disease, disorder, and/or condition (e.g., Hunters syndrome, Sanfilippo B syndrome). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Vector: As used herein, “vector” refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

### DETAILED DESCRIPTION

The present invention provides, among other things, methods and compositions for lysosomal targeting of a therapeutic protein (e.g., a lysosomal enzyme) through the use of a coupling moiety. In some embodiments, the present invention provides a targeted therapeutic comprising a therapeutic protein (e.g., a lysosomal enzyme) and a coupling moiety, wherein the coupling moiety is capable of binding a proprotein convertase protein and form a lysosomal delivery complex (LDC). In some embodiments, the coupling moiety is an antibody or binding fragment thereof. In some embodiments, the present invention provides an LDC comprising a lysosomal enzyme, wherein the LDC binds a non CI-MPR receptor. In some embodiments the LDC binds a one or more secondary binding proteins. As used herein, the term “secondary binding protein” is used to describe a protein which associates with a proprotein convertase protein through non-covalent binding. In some embodiments, the LDC binds to a secondary binding protein (e.g., membrane bound or transmembrane protein) via a cis-his rich domain (CHRD) to form a protein complex. In some embodiments, LDC binds to one or more secondary binding proteins selected from the group consisting of the low density lipoprotein receptor (LDLR), amyloid precursor-like protein 2 (APLP2), Dynamin, amyloid precursor protein (APP), autosomal recessive hypercholesterolemia (ARH) protein, low density lipoprotein receptor-related protein 8 (Lrp8), or combinations thereof.

Various aspects of the invention are described in further detail in the following subsections. The use of subsections is not meant to limit the invention. Each subsection may apply to any aspect of the invention. In this application, the use of “or” means “and/or” unless stated otherwise.

### Lysosomal Enzymes

The present invention may be used to target any therapeutic protein to a lysosome. In particular, the present invention may be used to target a lysosomal enzyme to a lysosome for the treatment of a lysosomal storage disease. According to the present invention, a lysosomal enzyme is contemplated to encompass any enzyme or protein, when targeted to the lysosome, is suitable for the treatment of a lysosomal storage disease. As non-limiting examples, particularly suitable lysosomal enzymes are acid alpha-glucosidase (GAA) protein, which is deficient in Pompe disease, and N-Acetylglucosaminidase (Naglu) protein, which is deficient in Sanfilippo Syndrome Type B disease. Additional exemplary lysosomal enzymes are shown in Table 3.

### GAA Protein

A suitable GAA protein according to the present invention can be any molecule that can substitute for naturally-occurring GAA protein activity or rescue one or more phenotypes or symptoms associated with GAA-deficiency. In some embodiments, a GAA protein suitable for the invention is a polypeptide having an N-terminus and C-terminus and an amino acid sequence substantially similar or identical to mature human GAA protein.

Typically, human GAA is produced as a precursor molecule that is processed to a mature form. This process generally occurs by removing the 27 amino acid signal peptide as the protein enters the endoplasmic reticulum. Typically, the form including the 27 amino acid signal peptide is referred to as Full-Length GAA protein, which contains 952 amino acids. The N-terminal 27 amino acids are cleaved as the Full-Length GAA protein enters the endoplasmic reticulum, resulting in the Precursor Form GAA Protein. The Precursor Form GAA Protein is then subsequently cleaved to remove a N-terminal pro-peptide sequence of 42 amino acids, to produce the Mature Form GAA protein (aa 70-952). Thus, it is contemplated that the N-terminal 27 amino acids that constitute the signal peptide and the N-terminal 42 amino acids that constitute the pro-peptide are generally not required for GAA protein activity. However, the use of the Full-Length GAA Protein (aa 1-952) and of the Precursor Form GAA Protein (aa 28-952) are also contemplated within the scope of the instant invention. The amino acid sequences of the Mature Form GAA Protein (SEQ ID NO:1); Precursor Form GAA Protein (SEQ ID NO:2) and Full-Length GAA Protein (SEQ ID NO:3) of a typical wild-type or naturally-occurring human GAA protein are shown in Table 1 below.

TABLE 1
Mature and Precursor GAA Protein

| | |
|---|---|
| Mature Form GAA | AHPGRPRAVPTQCDVPPNSRFDCAPDKAITQEQCEARGCCYIPAKQGLQGAQMG |
| Protein | QPWCFFPPSYPSYKLENLSSSEMGYTATLTRTTPTFFPKDILTLRLDVMMETEN |
| | RLHFTIKDPANRRYEVPLETPHVHSRAPSPLYSVEFSEEPFGVIVRRQLDGRVL |
| | LNTTVAPLFFADQFLQLSTSLPSQYITGLAEHLSPLMLSTSWTRITLWNRDLAP |
| | TPGANLYGSHPFYLALEDGGSAHGVFLLNSNAMDVVLQPSPALSWRSTGGILDV |
| | YIFLGPEPKSVVQQYLDVVGYPFMPPYWGLGFHLCRWGYSSTAITRQVVENMTR |
| | AHFPLDVQWNDLDYMDSRRDFTFNKDGFRDFPAMVQELHQGGRRYMMIVDPAIS |
| | SSGPAGSYRPYDEGLRRGVFITNETGQPLIGKVWPGSTAFPDFTNPTALAWWED |
| | MVAEFHDQVPFDGMWIDMNEPSNFIRGSEDGCPNNELENPPYVPGVVGGTLQAA |
| | TICASSHQFLSTHYNLHNLYGLTEAIASHRALVKARGTRPFVISRSTFAGHGRY |
| | AGHWTGDVWSSWEQLASSVPEILQFNLLGVPLVGADVCGFLGNTSEELCVRWTQ |
| | LGAFYPFMRNHNSLLSLPQEPYSFSEPAQQAMRKALTLRYALLPHLYTLFHQAH |
| | VAGETVARPLFLEFPKDSSTWTVDHQLLWGEALLITPVLQAGKAEVTGYFPLGT |
| | WYDLQTVPVEALGSLPPPPAAPREPAIHSEGQWVTLPAPLDTINVHLRAGYIIP |
| | LQGPGLTTTESRQQPMALAVALTKGGEARGELFWDDGESLEVLERGAYTQVIFL |
| | ARNNTIVNELVRVTSEGAGLQLQKVTVLGVATAPQQVLSNGVPVSNFTYSPDTK |
| | VLDICVSLLMGEQFLVSWC(SEQ ID NO: 1) |
| Precursor Form | GHILLHDFLLVPRELSGSSPVLEETHPAHQQGASRPGPRDAQAHPGRPRAVPTQ |
| GAA Protein | CDVPPNSRFDCAPDKAITQEQCEARGCCYIPAKQGLQGAQMGQPWCFFPPSYPS |
| | YKLENLSSSEMGYTATLTRTTPTFFPKDILTLRLDVMMETENRLHFTIKDPANR |
| | RYEVPLETPHVHSRAPSPLYSVEFSEEPFGVIVRRQLDGRVLLNTTVAPLFFAD |
| | QFLQLSTSLPSQYITGLAEHLSPLMLSTSWTRITLWNRDLAPTPGANLYGSHPF |
| | YLALEDGGSAHGVFLLNSNAMDVVLQPSPALSWRSTGGILDVYIFLGPEPKSVV |
| | QQYLDVVGYPFMPPYWGLGFHLCRWGYSSTAITRQVVENMTRAHFPLDVQWNDL |
| | DYMDSRRDFTFNKDGFRDFPAMVQELHQGGRRYMMIVDPAISSSGPAGSYRPYD |
| | EGLRRGVFITNETGQPLIGKVWPGSTAFPDFTNPTALAWWEDMVAEFHDQVPFD |
| | GMWIDMNEPSNFIRGSEDGCPNNELENPPYVPGVVGGTLQAATICASSHQFLST |
| | HYNLHNLYGLTEAIASHRALVKARGTRPFVISRSTFAGHGRYAGHWTGDVWSSW |
| | EQLASSVPEILQFNLLGVPLVGADVCGFLGNTSEELCVRWTQLGAFYPFMRNHN |
| | SLLSLPQEPYSFSEPAQQAMRKALTLRYALLPHLYTLFHQAHVAGETVARPLFL |
| | EFPKDSSTWTVDHQLLWGEALLITPVLQAGKAEVTGYFPLGTWYDLQTVPVEAL |
| | GSLPPPPAAPREPAIHSEGQWVTLPAPLDTINVHLRAGYIIPLQGPGLTTTESR |
| | QQPMALAVALTKGGEARGELFWDDGESLEVLERGAYTQVIFLARNNTIVNELVR |
| | VTSEGAGLQLQKVTVLGVATAPQQVLSNGVPVSNFTYSPDTKVLDICVSLLMGE |
| | QFLVSWC (SEQ ID NO: 2) |
| Full-Length GAA | MGVRHPPCSHRLLAVCALVSLATAALLGHILLHDFLLVPRELSGSSPVLEETHP |
| Protein | AHQQGASRPGPRDAQAHPGRPRAVPTQCDVPPNSRFDCAPDKAITQEQCEARGC |
| | CYIPAKQGLQGAQMGQPWCFFPPSYPSYKLENLSSSEMGYTATLTRTTPTFFPK |
| | DILTLRLDVMMETENRLHFTIKDPANRRYEVPLETPHVHSRAPSPLYSVEFSEE |
| | PFGVIVRRQLDGRVLLNTTVAPLFFADQFLQLSTSLPSQYITGLAEHLSPLMLS |
| | TSWTRITLWNRDLAPTPGANLYGSHPFYLALEDGGSAHGVFLLNSNAMDVVLQP |
| | SPALSWRSTGGILDVYIFLGPEPKSVVQQYLDVVGYPFMPPYWGLGFHLCRWGY |
| | SSTAITRQVVENMTRAHFPLDVQWNDLDYMDSRRDFTFNKDGFRDFPAMVQELH |
| | QGGRRYMMIVDPAISSSGPAGSYRPYDEGLRRGVFITNETGQPLIGKVWPGSTA |
| | FPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEALASHRALVKARGTR |
| | PFVISRSTFAGHGRYAGHWTGDVWSSWEQLASSVPEILQFNLLGVPLVGADVCG |
| | FLGNTSEELCVRWTQLGAFYPFMRNHNSLLSLPQEPYSFSEPAQQAMRKALTLR |
| | YALLPHLYTLFHQAHVAGETVARPLFLEFPKDSSTWTVDHQLLWGEALLITPVL |
| | QAGKAEVTGYFPLGTWYDLQTVPVEALGSLPPPPAAPREPAIHSEGQWVTLPAP |
| | LDTINVHLRAGYIIPLQGPGLTTTESRQQPMALAVALTKGGEARGELFWDDGES |
| | LEVLERGAYTQVIFLARNNTIVNELVRVTSEGAGLQLQKVTVLGVATAPQQVLS |
| | NGVPVSNFTYSPDTKVLDICVSLLMGEQFLVSWC (SEQ ID NO: 3) |

Thus, in some embodiments, GAA protein suitable for the present invention is a human Mature Form GAA Protein (SEQ ID NO:1). In some embodiments, a suitable GAA protein may be a homologue or an orthologue of human Mature Form GAA Protein from a different species (e.g., mouse, rat, sheep, pig, dog, etc.). In other embodiments, a suitable GAA protein may be a functional variant of human Mature Form GAA Protein. A functional variant Mature Form GAA Protein may be a modified human Mature Form GAA Protein containing one or more amino acid substitutions, deletions, and/or insertions as compared to a wild-type or naturally-occurring human Mature Form GAA Protein (e.g., SEQ ID NO:1), while retaining substantial GAA protein activity. Thus, in some embodiments, a GAA protein suitable for the present invention is substantially homologous to human Mature Form GAA Protein (SEQ ID NO:1). In some embodiments, a GAA protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:1. In some embodiments, a GAA protein suitable for the present invention is substantially identical to human Mature Form GAA Protein (SEQ ID NO:1). In some embodiments, a GAA protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:1. In some embodiments, a GAA protein suitable for the present invention contains a fragment or a portion of human Mature Form GAA Protein.

Alternatively, a GAA protein suitable for the present invention is a human Precursor Form GAA Protein (SEQ ID NO:2). In some embodiments, a GAA protein suitable may be a homologue or an orthologue of human Precursor Form GAA Protein from a different species (e.g., mouse, rat, sheep, pig, dog, etc.). In some embodiments, a suitable GAA protein is a functional variant of a human Precursor Form GAA Protein, containing one or more amino acid substitutions, deletions, and/or insertions as compared to a wild-type or naturally-occurring human Precursor Form GAA Protein (e.g., SEQ ID NO:2), while retaining substantial GAA protein activity. Thus, in some embodiments, a GAA protein suitable for the present invention is substantially homologous to human Precursor Form GAA Protein (SEQ ID NO:2). In some embodiments, a GAA protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:2. In some embodiments, a GAA protein suitable for the present invention is substantially identical to SEQ ID NO:2. In some embodiments, a GAA protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:2. In some embodiments, a GAA protein suitable for the present invention contains a fragment or a portion of human Precursor Form GAA Protein. As used herein, a Precursor Form GAA Protein typically contains a pro-peptide sequence.

Alternatively, a GAA protein suitable for the present invention is a human Full-Length GAA Protein (SEQ ID NO:3). In some embodiments, a GAA protein suitable may be a homologue or an orthologue of Full-Length GAA Protein from a different species (e.g., mouse, rat, sheep, pig, dog, etc.). In some embodiments, a suitable GAA protein is a functional variant of human Full-Length GAA Protein, containing one or more amino acid substitutions, deletions, and/or insertions as compared to a wild-type or naturally-occurring full length GAA protein (e.g., SEQ ID NO:3), while retaining substantial GAA protein activity. Thus, in some embodiments, a GAA protein suitable for the present invention is substantially homologous to human Full-Length GAA Protein (SEQ ID NO:3). In some embodiments, a GAA protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:3. In some embodiments, a GAA protein suitable for the present invention is substantially identical to SEQ ID NO:3. In some embodiments, a GAA protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:3. In some embodiments, a GAA protein suitable for the present invention contains a fragment or a portion of human Full-Length GAA Protein. As used herein, a Full-Length GAA Protein typically contains a signal peptide sequence and a pro-peptide sequence.

### Naglu Protein

A suitable Naglu protein according to the present invention can be any molecule that can substitute for naturally-occurring Naglu protein activity or rescue one or more phenotypes or symptoms associated with Naglu-deficiency. In some embodiments, a Naglu protein suitable for the invention is a polypeptide having an N-terminus and C-terminus and an amino acid sequence substantially similar or identical to mature human Naglu protein.

Typically, human Naglu is produced as a precursor molecule that is processed to a mature form. This process generally occurs by removing the 23 amino acid signal peptide as the protein enters the endoplasmic reticulum. Typically, the precursor form is also referred to as full-length precursor or full-length Naglu protein, which contains 743 amino acids. The N-terminal 23 amino acids are cleaved as the precursor protein enters the endoplasmic reticulum, resulting in a mature form. Thus, it is contemplated that the N-terminal 23 amino acids is generally not required for the Naglu protein activity. However, the use of the full-length precursor of the Naglu protein is also contemplated within the scope of the instant invention. The amino acid sequences of the mature form (SEQ ID NO:4) and full-length precursor (SEQ ID NO:5) of a typical wild-type or naturally-occurring human Naglu protein are shown in Table 2 below.

TABLE 2
Mature and Precursor Naglu Protein

| | |
|---|---|
| Mature Form of | DEAREAAAVRALVARLLGPGPAADFSVSVERALAAKPGLDTYSLGGGGAARVRV |
| Naglu | RGSTGVAAAAGLHRYLRDFCGCHVAWSGSQLRLPRPLPAVPGELTEATPNRYRY |
| | YQNVCTQSYSFVWWDWARWEREIDWMALNGINLALAWSGQEAIWQRVYLALGLT |
| | QAEINEFFTGPAFLAWGRMGNLHTWDGPLPPSWHIKQLYLQHRVLDQMRSFGMT |
| | PVLPAFAGHVPEAVTRVFPQVNVTKMGSWGHFNCSYSCSFLLAPEDPIFPIIGS |
| | LFLRELIKEFGTDHIYGADTFNEMQPPSSEPSYLAAATTAVYEAMTAVDTEAVW |
| | LLQGWLFQHQPQFWGPAQIRAVLGAVPRGRLLVLDLFAESQPVYTRTASFQGQP |
| | FIWCMLHNFGGNHGLFGALEAVNGGPEAARLFPNSTMVGTGMAPEGISQNEVVY |
| | SLMAELGWRKDPVPDLAAWVTSFAARRYGVSHPDAGAAWRLLLRSVYNCSGEAC |
| | RGHNRSPLVRRPSLQMNTSIWYNRSDVFEAWRLLLTSAPSLATSPAFRYDLLDL |
| | TRQAVQELVSLYYEEARSAYLSKELASLLRAGGVLAYELLPALDEVLASDSRFL |
| | LGSWLEQARAAAVSEAEADFYEQNSRYQLTLWGPEGNILDYANKQLAGLVANYY |
| | TPRWRLFLEALVDSVAQGIPFQQHQFDKNVFQLEQAFVLSKQRYPSQPRGDTVD |
| | LAKKIFLKYYPRWVAGSW (SEQ ID NO: 4) |
| Full-Length | MEAVAVAAAVGVLLLAGAGGAAGDEAREAAAVRALVARLLGPGPAADFSVSVER |
| Precursor/Full- | ALAAKPGLDTYSLGGGGAARVRVRGSTGVAAAAGLHRYLRDFCGCHVAWSGSQL |
| Length Naglu Protein | RLPRPLPAVPGELTEATPNRYRYYQNVCTQSYSFVWWDWARWEREIDWMALNGI |
| | NLALAWSGQEAIWQRVYLALGLTQAEINEFFTGPAFLAWGRMGNLHTWDGPLPP |
| | SWHIKQLYLQHRVLDQMRSFGMTPVLPAFAGHVPEAVTRVFPQVNVTKMGSWGH |
| | FNCSYSCSFLLAPEDPIFPIIGSLFLRELIKEFGTDHIYGADTFNEMQPPSSEP |
| | SYLAAATTAVYEAMTAVDTEAVWLLQGWLFQHQPQFWGPAQIRAVLGAVPRGRL |
| | LVLDLFAESQPVYTRTASFQGQPFIWCMLHNFGGNHGLFGALEAVNGGPEAARL |
| | FPNSTMVGTGMAPEGISQNEVVYSLMAELGWRKDPVPDLAAWVTSFAARRYGVS |
| | HPDAGAAWRLLLRSVYNCSGEACRGHNRSPLVRRPSLQMNTSIWYNRSDVFEAW |
| | RLLLTSAPSLATSPAFRYDLLDLTRQAVQELVSLYYEEARSAYLSKELASLLRA |
| | GGVLAYELLPALDEVLASDSRFLLGSWLEQARAAAVSEAEADFYEQNSRYQLTL |
| | WGPEGNILDYANKQLAGLVANYYTPRWRLFLEALVDSVAQGIPFQQHQFDKNVF |
| | QLEQAFVLSKQRYPSQPRGDTVDLAKKIFLKYYPRWVAGSW (SEQ ID |
| | NO: 5) |

Thus, in some embodiments, Naglu protein suitable for the present invention is a mature human Naglu protein (SEQ ID NO:4). In some embodiments, a suitable Naglu protein may be a homologue or an orthologue of the mature human Naglu protein from a different species (e.g., mouse, rat, sheep, pig, dog, etc.). In other embodiments, a suitable Naglu protein may be a functional variant of the mature human Naglu protein. A functional variant of the mature human Naglu protein may be a modified mature human Naglu protein containing one or more amino acid substitutions, deletions, and/or insertions as compared to a wild-type or naturally-occurring Naglu protein (e.g., SEQ ID NO:4), while retaining substantial Naglu protein activity. Thus, in some embodiments, a Naglu protein suitable for the present invention is substantially homologous to mature human Naglu protein (SEQ ID NO:4). In some embodiments, a Naglu protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:4. In some embodiments, a Naglu protein suitable for the present invention is substantially identical to mature human Naglu protein (SEQ ID NO:4). In some embodiments, a Naglu protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:4. In some embodiments, a Naglu protein suitable for the present invention contains a fragment or a portion of a mature Naglu protein.

Alternatively, a Naglu protein suitable for the present invention is a full-length Naglu protein (SEQ ID NO:5). In some embodiments, a Naglu protein suitable may be a homologue or an orthologue of the full-length human Naglu protein from a different species (e.g., mouse, rat, sheep, pig, dog, etc.). In some embodiments, a suitable Naglu protein is a functional variant of the full-length human Naglu protein, containing one or more amino acid substitutions, deletions, and/or insertions as compared to a wild-type or naturally-occurring full-length Naglu protein (e.g., SEQ ID NO:5), while retaining substantial Naglu protein activity. Thus, in some embodiments, a Naglu protein suitable for the present invention is substantially homologous to full-length human Naglu protein (SEQ ID NO:5). In some embodiments, a Naglu protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:5. In some embodiments, a Naglu protein suitable for the present invention is substantially identical to SEQ ID NO:5. In some embodiments, a Naglu protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:5. In some embodiments, a Naglu protein suitable for the present invention contains a fragment or a portion of a full-length Naglu protein. As used herein, a full-length Naglu protein typically contains a signal peptide sequence.

### Additional Lysosomal Enzymes

The present invention may be used to deliver any lysosomal enzymes that can be used to treat any lysosomal storage diseases, in particular those lysosomal storage diseases having skeletal musce, kidney and/or CNS etiology and/or symptoms, including, but are not limited to, aspartylglucosaminuria, cholesterol ester storage disease, Wolman disease, cystinosis, Danon disease, Fabry disease, Farber lipogranulomatosis, Farber disease, fucosidosis, galactosialidosis types I/II, Gaucher disease types I/II/III, globoid cell leukodystrophy, Krabbe disease, glycogen storage disease II, Pompe disease, GM1-gangliosidosis types I/II/III, GM2-gangliosidosis type I, Tay Sachs disease, GM2-gangliosidosis type II, Sandhoff disease, GM2-gangliosidosis, α-mannosidosis types I/II, .beta.-mannosidosis, metachromatic leukodystrophy, mucolipidosis type I, sialidosis types I/II, mucolipidosis types II/III, I-cell disease, mucolipidosis type IIIC pseudo-Hurler polydystrophy, mucopolysaccharidosis type I, mucopolysaccharidosis type II, mucopolysaccharidosis type MA, Sanfilippo syndrome, mucopolysaccharidosis type IIIB, mucopolysaccharidosis type IIIC, mucopolysaccharidosis type HID, mucopolysaccharidosis type IVA, Morquio syndrome, mucopolysaccharidosis type IVB, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, CLN2 Batten diseae, Niemann-Pick disease types A/B, Niemann-Pick disease type C1, Niemann-Pick disease type C2, pycnodysostosis, Schindler disease types I/II, Gaucher disease and sialic acid storage disease.

A detailed review of the genetic etiology, clinical manifestations, and molecular biology of the lysosomal storage diseases are detailed in Scriver et al., eds., The Metabolic and Molecular Basis of Inherited Disease, 7.sup.th Ed., Vol. II, McGraw Hill, (1995). Thus, the enzymes deficient in the above diseases are known to those of skill in the art, some of these are exemplified in Table 3 below:

TABLE 3
Enzymes Associated With Lysosomal Storage Disease

| Disease Name | Enzyme Deficiency | Substance Stored |
|---|---|---|
| Pompe Disease | Acid-a1,4-Glucosidase | Glycogen α-1-4 linked |
| | | Oligosaccharides |
| GM1 Gangliodsidosis | β-Galactosidase | GM₁ Gangliosides |
| Tay-Sachs Disease | β-Hexosaminidase A | GM₂ Ganglioside |
| GM2 Gangliosidosis: AB | GM₂ Activator Protein | GM₂ Ganglioside |
| Variant | | |
| Sandhoff Disease | β-Hexosaminidase A&B | GM₂ Ganglioside |
| Fabry Disease | α-Galactosidase A | Globosides |
| Gaucher Disease | Glucocerebrosidase | Glucosylceramide |
| Metachromatic | Arylsulfatase A | Sulphatides |
| Leukodystrophy | | |
| Krabbe Disease | Galactosylceramidase | Galactocerebroside |
| Niemann Pick, Types A & B | Acid Sphingomyelinase | Sphingomyelin |
| Niemann-Pick, Type C | Cholesterol Esterification Defect | Sphingomyelin |
| Niemann-Pick, Type D | Unknown | Sphingomyelin |
| Farber Disease | Acid Ceramidase | Ceramide |
| Wolman Disease | Acid Lipase | Cholesteryl Esters |
| Hurler Syndrome (MPS IH) | α-L-Iduronidase | Heparan & Dermatan Sulfates |
| Scheie Syndrome (MPS IS) | α-L-Iduronidase | Heparan & Dermatan, Sulfates |
| Hurler-Scheie (MPS IH/S) | α-L-Iduronidase | Heparan & Dermatan Sulfates |
| Hunter Syndrome (MPS II) | Iduronate Sulfatase | Heparan & Dermatan Sulfates |
| Sanfilippo A (MPS IIIA) | Heparan N-Sulfatase | Heparan Sulfate |
| Sanfilippo B (MPS IIIB) | α-N- | Heparan Sulfate |
| | Acetylglucosaminidase | |
| Sanfilippo C (MPS IIIC) | Acetyl-CoA- | Heparan Sulfate |
| | Glucosaminide | |
| | Acetyltransferase | |
| Sanfilippo D (MPS IIID) | N-Acetylglucosamine-6- | Heparan Sulfate |
| | Sulfatase | |
| Morquio B (MPS IVB) | β-Galactosidase | Keratan Sulfate |
| Maroteaux-Lamy (MPS VI) | Arylsulfatase B | Dermatan Sulfate |
| Sly Syndrome (MPS VII) | β-Glucuronidase | |
| α-Mannosidosis | α-Mannosidase | Mannose/Oligosaccharides |
| β-Mannosidosis | β-Mannosidase | Mannose/Oligosaccharides |
| Fucosidosis | α-L-Fucosidase | Fucosyl/Oligosaccharides |
| Aspartylglucosaminuria | N-Aspartyl-β- | Aspartylglucosamine |
| | Glucosaminidase | Asparagines |
| Sialidosis (Mucolipidosis I) | α-Neuraminidase | Sialyloligosaccharides |
| Galactosialidosis | Lysosomal Protective | Sialyloligosaccharides |
| (Goldberg Syndrome) | Protein Deficiency | |
| Schindler Disease | α-N-Acetyl- | |
| | Galactosaminidase | |
| Mucolipidosis II (I-Cell Disease) | N-Acetylglucosamine-1- | Heparan Sulfate |
| | Phosphotransferase | |
| Mucolipidosis III (Pseudo- | Same as ML II | |
| Hurler Polydystrophy) | | |
| Cystinosis | Cystine Transport Protein | Free Cystine |
| Salla Disease | Sialic Acid Transport | Free Sialic Acid |
| | Protein | and Glucuronic Acid |
| Infantile Sialic Acid | Sialic Acid Transport | Free Sialic Acid |
| Storage Disease | Protein | and Glucuronic Acid |
| Infantile Neuronal Ceroid | Palmitoyl-Protein | Lipofuscins |
| Lipofuscinosis | Thioesterase | |
| Mucolipidosis IV | Unknown | Gangliosides & |
| | | Hyaluronic Acid |
| Prosaposin | Saposins A, B, C or D | |

In some embodiments, a suitable lysosomal enzyme may be a naturally occurring lysosomal enzyme. In some embodiments, a suitable lysosomal enzyme may be a recombinant version of a naturally occurring lysosomal enzyme.

In some embodiments, a lysosomal enzyme suitable for the invention may have a wild-type or naturally occurring sequence. In some embodiments, a lysosomal enzyme suitable for the invention may have a modified sequence having substantial homology or identify to the wild-type or naturally-occurring sequence (e.g., having at least 50%, 55%, 60%, 65%, 70%, 75%, 80%, 85%, 90%, 95%, 98% sequence identity to the wild-type or naturally-occurring sequence).

### Coupling Moiety

As used herein, the term “coupling moiety” refers to an agent that is associated with a therapeutic protein, through ionic or covalent bonding, and is capable of binding to an antigen or biological target to facilitate lysosomal targeting. In some embodiments, the coupling moiety comprises a protein. In some embodiments, the coupling moiety is or comprises a naturally occurring protein. In some embodiments, the coupling moiety is derived from a cell. In some embodiments, the coupling moiety is a synthetic or chemically synthesized protein. In some embodiments, coupling moieties are comprised of natural amino acids. In other embodiments, the coupling moiety comprises one or more unnatural amino acids. In some embodiments, the coupling moiety is comprised of a combination of natural and unnatural amino acids. In some embodiments, the coupling moiety is comprised of one, two or more polypeptide chains that are covalently or non-covalently associated. In some embodiments, the coupling moiety may be linked to, or part of, a longer polypeptide chain, so long as the coupling moiety retains its three-dimensional structure and arrangement for interaction. In some specific embodiments, the coupling moiety may be appended to the N- or C-termini of another polypeptide sequence, such as a therapeutic protein, via a translational fusion.

In some embodiments, the coupling moiety is a protein that functions similarly to an antibody and is able to bind to a specific antigen to form a complex and may or may not elicit a biological response (e.g., agonize or antagonize a particular biological activity.) In some embodiments, the coupling moiety is an antibody. In some embodiments, the coupling moiety is or comprises a “full length” antibody, in that it contains two heavy chains and two light chains, optionally associated by disulfide bonds as occurs with naturally-produced antibodies.

In some embodiments, the coupling moiety is or comprises a fragment of a full-length antibody in that is contains some, but not all of the sequences found in a full-length antibody. As used herein, an “antibody fragment” includes a portion of an intact antibody, such as, for example, the antigen-binding or variable region of an antibody. Examples of antibody fragments include Fab, Fab′, F(ab′)₂, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)₂ fragment, scFv fragment, Fv fragment, dsFv diabody, dAb fragment, Fd′ fragment, Fd fragment, and an isolated complementarity determining region (CDR) region.

In some embodiments, a provided coupling moiety is or comprises a VHH (i.e., an antigen-specific VHH) antibody that comprises only a heavy chain. In some embodiments the VHH is derived from a llama or other camelid antibody (e.g., a camelid IgG2 or IgG3, or a CDR-displaying frame from such camelid Ig). In some embodiments a VHH is derived from a shark.

In some embodiments, a coupling moiety comprises one or more “Mini-antibodies” or “minibodies”. Minibodies are sFv polypeptide chains which include oligomerization domains at their C-termini, separated from the sFv by a hinge region. Pack et al. (1992) Biochem 31:1579-1584. The oligomerization domain comprises self-associating a-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J Immunology 149B:120-126.

In some embodiments, a coupling moiety comprises one or more antibody-like binding scaffold proteins. For example, in some embodiments, one or more CDRs arising from an antibody may be grafted onto a protein scaffold. In general, protein scaffolds may meet the greatest number of the following criteria: (Skerra A., J. Mol. Recogn., 2000, 13:167-187): good phylogenetic conservation; known three-dimensional structure (as, for example, by crystallography, NMR spectroscopy or any other technique known to a person skilled in the art); small size; few or no post-transcriptional modifications; and/or easy to produce, express and purify. The origin of such protein scaffolds can be, but is not limited to, fibronectin (e.g., fibronectin type III domain 10), lipocalin, anticalin (Skerra A., J. Biotechnol., 2001, 74(4):257-75), protein Z arising from domain B of protein A of Staphylococcus aureus, thioredoxin A or proteins with a repeated motif such as the “ankyrin repeat” (Kohl et al., PNAS, 2003, vol. 100, No. 4, 1700-1705), the “armadillo repeat”, the “leucine-rich repeat” and the “tetratricopeptide repeat”. For example, anticalins or lipocalin derivatives are described in US Patent Publication Nos. 20100285564, 20060058510, 20060088908, 20050106660, and PCT Publication No. WO2006/056464, incorporated herein by reference. Scaffolds derived from toxins such as, for example, toxins from scorpions, insects, plants, mollusks, etc., and the protein inhibitors of neuronal NO synthase (PIN) may also be used in accordance with the present invention. In some embodiments, the coupling moiety is a scaffold protein such as, but is not limited to, protein A, lipoclins, ankryin consensus repeat domain, thioredoxin, adnectin, anticalins, centyrin, avimer domains, ubiquitin, zinc finger DNA-binding proteins (ZEPs), or IgNARs. In some embodiments, a coupling moiety is a scaffold protein, in which the scaffold protein is engineered to display one or more CDRs.

In some embodiments, a provided coupling moiety is or comprises a cystine-knot miniprotein. In some embodiments, a provided coupling moiety is or comprises an avibody (diabody, tribody, tetrabody). In some embodiments, a provided coupling moiety is or comprises a Scorpion, wherein the Scorpion structure comprises two binding moieties separated by an immunoglobulin Fc domain. In some embodiments, the provided coupling moiety is a stapled peptide.

In some embodiments, provided coupling moieties include one or more antibody-like binding peptidomimetics. Liu et al. Cell Mol Biol (Noisy-le-grand). 2003 March; 49(2):209-16 describe “antibody like binding peptidomimetics” (ABiPs), which are peptides that act as pared-down antibodies and have certain advantages of longer serum half-life as well as less cumbersome synthesis methods. Likewise, in some aspects, antibody-like molecules are cyclic or bicyclic peptides. For example, methods for isolating antigen-binding bicyclic peptides (e.g., by phage display) and for using the such peptides are provided in U.S. Patent Publn. No. 20100317547, incorporated herein by reference.

In some specific embodiments the coupling moiety is associated with a lysosomal enzyme to form a targeted therapeutic. In some embodiments, a coupling moiety of the targeted therapeutic is capable of binding to a proprotein convertase protein (e.g., PCSK9) to form a lysosmal delivery complex (LDC).

### Proprotein Convertases

Mammalian proprotein convertases constitute a secretory serine protease family composed of nine members related to bacterial subtilisin and yeast kexin. The catalytic domains of seven members of this family (PC1/3; PC2; Furin; PC4; PC5/6; PACE4 and PC7) exhibit homology to the catalytic domain of yeast kexin, and they are known to cleave after basic residues in target proteins. The eighth member, SKI-1/S1P, shows strong homology to bacterial pyrolysin and, similar to the other 7 family members, is known to cleave after basic residues in target proteins. Finally, the last member, PCSK9, shows homology to fungal proteinase K and undergoes autoproteolytic cleavage at the (V/I)FAQ motif in the endoplasmic reticulum. Like many other proteases, these proprotein convertases are synthesized as inactive zymogens that carry an N-terminal propeptide. It is thought that this propeptide facilitates proper folding of the convertase, and that it functions as a natural inhibitor of the enzyme until it is cleaved off.

Among the nine family members, five PCs (Furin, PC5/6; PACE4, SKI-1/S1P and PCSK9) have been shown to play a central role in regulating sterols and/or lipid metabolism. This is especially true for PCSK9, whose over-activity/gain-of-function results in Familial Hypercholesterolemia (FH). PCSK9 is highly expressed in the liver and produced as a pre-protein that undergoes autoproteolytic cleavage during passage through the secretory pathyway. During this process, the C-terminus of the N-terminal propeptide occupies PCSK9's catalytic pocket, inhibiting its proteolytic activity and blocking access to other exogenous substrates.

PCSK9 also binds to the EGF-A domain of the LDL receptor through part of its catalytic domain to form a non-covalent protein complex, which is internalized by endocytosis and targeted for degradation in the acidic compartment of the lysosome.

Data suggest, that while the PCSK9-LDLR complex is necessary for LDL receptor (LDLR) recycling and removal of LDL from the extracellular space, it is not required for PCSK9 endocytosis to the lysosome. Several studies have shown that disruption of PCSK9 binding to LDLR, through mutations within its catalytic domain or via the use of blocking antibodies does not impede PCSK9 cellular internalization. This suggests that alternative mechanisms exist by which PCSK9 is internalized. In particular, it has been suggested that lysosomal targeting and function of PCSK9 relies on its C-terminal Cys-His-rich domain (CHRD), a region which allows for non-covalent binding with various membrane bound protein such as: amyloid precursor-like protein 2 (APLP2), Dynamin, amyloid precursor protein (APP), autosomal recessive hypercholesterolemia (ARH) protein, low density lipoprotein receptor-related protein 8 (Lrp8) and Annexin A2 (LoSurdo et al., EMBO 12:1300-1305 (2011); Ni et al., J. Biol. Chem. 285:12882-12891 (2010); Saavedra et al., J. Biol. Chem. 287:43492-43501 (2012); DeVay et al., J. Biol. Chem. 288:10805-10818 (2013); and Chaparro-Riggers et al., J. Biol. Chem. 287:11090-11097 (2012); the contents of all of which are hereby incorporated by reference.)

In some specific embodiments a coupling moiety is capable of binding to one or more pre-selected binding sites within a proprotein convertase. In some embodiments, a coupling moiety is capable of binding to any proprotein convertase molecule, fragment or portion thereof (e.g. a motif or domain) capable of binding, directly or indirectly, to the LDL receptor (LDLR). In some embodiments, the proprotein convertase molecule or fragment thereof, is capable of binding, directly or indirectly, to a secondary binding protein selected from the group consisting of amyloid precursor-like protein 2 (APLP2), Dynamin, amyloid precursor protein (APP), autosomal recessive hypercholesterolemia (ARH) protein, low density lipoprotein receptor-related protein 8 (Lrp8) and Annexin A. As used herein, binding to a secondary binding protein typically refers to a physiologically meaningful binding. For example, a physiologically meaningful binding typically has a dissociation constant (Kd) no greater than 10⁻⁷ under physiological conditions (e.g., pH 6-8, and in particular, pH 7.4).

In some embodiments, the proprotein convertase is a mammalian convertase. In some embodiments, the proprotein convertase is selected from the group consisting of PC1/3; PC2; Furin; PC4; PC5/6; PACE4, PC7, SKI-1/S1P and PCSK9. In some embodiments, the proprotein convertase is PCSK9.

In some embodiments, the coupling moiety is capable of binding to a selected binding site of PCSK9. In some embodiments, the coupling moiety is capable of binding to that site of PCSK9 that binds to the EGF-A domain of LDLR. In some embodiments, the coupling moiety binds a site within the catalytic domain comprising D186, H226 and/or 5386 of the wildtype PCSK9 amino acid sequence. In some embodiments, the coupling moiety is capable of binding to the LDLR binding site on PCSK9. In some embodiments, the coupling agent binds a site within the LDLR binding site comprising R194 and/or F379 of the wildtype PCSK9 amino acid sequence. In some specific embodiments, the coupling moiety is capable of binding to the CHRD domain of PCSK9. In some embodiments, the coupling moiety is capable of binding to one or more binding sites of PCSK9 selected from the group consisting of LDLR binding site, CHRD domain, autocatalytic site and combinations thereof.

In some specific embodiments, binding of the coupling moiety to the proprotein convertase (e.g., PCSK9) alters binding of the proprotein convertase within the LDC to one or more secondary binding proteins. In some embodiments, a coupling moiety is an agent that is able to bind to PCSK9 and compete with binding to a secondary binding protein (e.g., LDL receptor), such that binding between the PCSK9 and a secondary protein is reduced by at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, or at least 20 fold. In some embodiments, a coupling moiety is an agent that is able to bind to a proprotein convertase (e.g. PCSK9) and completely disrupt binding to a secondary binding protein. In some embodiments, a coupling moiety is an agent that is able to enhance binding of a proprotein convertase (e.g., PCSK9) to a secondary binding protein, (e.g., Amyloid Precursor-like Protein 2 (APLP2), Dynamin, Amyloid Precursor Protein (APP), Autosomal Recessive Hypercholesterolemia (ARH) protein, or Low Density Lipoprotein Receptor-related Protein 8 (Lrp8)), such that binding between a proprotein convertase (e.g., PCSK9) and a secondary binding protein is enhanced by at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, or at least 20 fold.

### Association Between Lysosomal Enzyme and Lysosomal Coupling Moiety

A lysosomal enzyme and a coupling moiety can be associated, directly or indirectly. In some embodiments, a lysosomal enzyme and a coupling moiety are non-covalently associated. The association is typically stable at or about pH 7.4. For example, a coupling moiety can be biotinylated and bind avidin associated with a lysosomal enzyme. In some embodiments, a coupling moiety and a lysosomal enzyme are crosslinked to each other (e.g., using a chemical crosslinking agent).

In some embodiments, a coupling moiety is fused to a lysosomal enzyme as a fusion protein. The coupling moiety can be at the amino-terminus of the fusion protein, the carboxy-terminus, or can be inserted within the sequence of the lysosomal enzyme at a position where the presence of the coupling moiety does not unduly interfere with the therapeutic activity of the enzyme. Where a lysosomal enzyme is a heteromeric protein, one or more of the subunits can be associated with a coupling moeity.

### Linker or Spacer

A coupling moiety can be fused to the N-terminus or C-terminus of a polypeptide encoding a lysosomal enzyme, or inserted internally. The coupling moiety can be fused directly to the lysosomal enzyme polypeptide or can be separated from the lysosomal enzyme polypeptide by a linker or a spacer. An amino acid linker or spacer is generally designed to be flexible or to interpose a structure, such as an alpha-helix, between the two protein moieties. A linker or spacer can be relatively short, such as a GGG or a poly “GAG” sequence GGGGGAAAAAGGGGG (SEQ ID NO:6), a “GAP” sequence of GAP (SEQ ID NO:7), a “PolyGP” sequence of GGGGGP (SEQ ID NO:8), or can be longer, such as, for example, 10-50 (e.g., 10-20, 10-25, 10-30, 10-35, 10-40, 10-45, 10-50) amino acids in length. In some embodiments, various short linker sequences can be present in tandem repeats. For example, a suitable linker may contain the “GAG” amino acid sequence of GGGGGAAAAAGGGGG (SEQ ID NO:6) present in tandem repeats. In some embodiments, such a linker may further contain one or more “GAP” sequences, that frame the “GAG” sequence of GGGGGAAAAAGGGGG (SEQ ID NO:6). For example, in some embodiments a GAG2 linker may be used, which contains two tandem “GAG” repeats, each framed by a “GAP” sequence, such as GAPGGGGGAAAAAGGGGGGAPGGGGGAAAAAGGGGGGAP (SEQ ID NO:9). In some embodiments a GAG3 linker may be used, which contains three tandem “GAG” repeats, each framed by two “GAP” sequences, such as GAPGGGGGAAAAAGGGGGGAPGGGGGAAAAAGGGGGGAPGGGGGAAAAAGGGGG GAP (SEQ ID NO:10).

In some embodiments, a suitable linker or spacer may contain a sequence at least 50%, 55%, 60%, 65%, 70%, 75%, 80%, 85%, 90%, 95%, 98%, or 99% identical to any of the linker sequences described herein, including, but not limited to, SEQ ID NO:6, SEQ ID NO:7, SEQ ID NO:8, SEQ ID NO:9, or SEQ ID NO:10.

Additional linkers or spacers suitable for the invention are known in the art including those described in WO 2012122042, entitled “PEPTIDE LINKERS FOR POLYPEPTIDE COMPOSITIONS AND METHODS FOR USING SAME”, which is incorporated by reference in its entirety.

In some embodiments of the present invention, a suitable linker or spacer may contain a lysosomal protease cleavage site.

It is contemplated that the association between a lysosomal enzyme and a coupling moiety according to the present invention does not substantially alter enzyme activity. In some embodiments, the targeted therapeutic has an enzyme activity that is substantially similar or enhanced when compared to the corresponding native enzyme. In some embodiments, the enzyme activity of a targeted therapeutic retains at least about 60%, 65%, 70%, 75%, 80%, 85%, 90%, 95%, 98%, 99%, or 100% enzymatic activity as compared to the native enzyme. In some embodiments, the enzyme activity of a targeted therapeutic is enhanced by at least about 5%, 10%, 15%, 20%, 25%, 30%, 35%, 40%, 45%, 50%, 55%, 60%, 70%, 80%, 90% or 100% compared to the native enzyme.

In some embodiments, a targeted therapeutic of the present invention comprises a GAA or Naglu protein fused to a coupling moiety. In some embodiments, the GAA or Naglu protein has a Km for a known substrate of at least about 0.10 nM (e.g., at least about 0.15 nM, 0.20 nM, 0.25 nM, 0.30 nM, or 0.35 nM).

### Lysosomal Delivery Complex (LDC)

It is also contemplated that in some embodiments, the targeted therapeutic of the present invention permits substantial binding between the coupling moiety and a proprotein convertase (e.g., PCSK9) to form a LDC. In some embodiments, the targeted therapeutic of the present invention may be engineered to permit substantial binding between the coupling moiety and proprotein convertase protein, to promote binding to one or more secondary proteins. In some embodiments, the targeted therapeutic is engineered to promote binding to one or more secondary proteins, such as, but not limited to, amyloid precursor-like protein 2 (APLP2), Dynamin, amyloid precursor protein (APP), autosomal recessive hypercholesterolemia (ARH) protein, low density lipoprotein receptor-related protein 8 (Lrp8) and Annexin A, while reducing or completely blocking binding to LDLR. In some embodiments, the level of LDC binding to one or more secondary binding proteins may be tested using any of a variety of well-known binding assays, such as, but not limited to, radiolabeled run on assay, radiolabeled binding assay, ELISA, Surface Plasmone Resonance and Isothermal Titration calorimetry. In some embodiments, the level of targeted lysosomal delivery of the targeted therapeutic may be evaluated by assaying for cellular uptake of a targeted therapeutic.

In some embodiments, a targeted therapeutic has an average association constant (ka [1/Ms]) of at least about 1.0×10⁵ (e.g., at least about 1.0×10⁶, 1.0×10⁷, 1.0×10⁸, 1.0×10⁹) for a proprotein convertase protein. In some embodiments, the LDC has an average association constant (ka [1/Ms]) of at least about 1.0×10⁵ (e.g., at least about 1.0×10⁶, 1.0×10⁷, 1.0×10⁸, 1.0×10⁹) for one or more secondary binding proteins, such as, but not limited to amyloid precursor-like protein 2 (APLP2), Dynamin, amyloid precursor protein (APP), autosomal recessive hypercholesterolemia (ARH) protein, low density lipoprotein receptor-related protein 8 (Lrp8), LDLR and Annexin A.

In some embodiments, the cellular uptake of a targeted therapeutic according to the present invention has a Kd of at least about 1.0e+2 nM (e.g., at least about 1.0e+3 nM, 1.0e+4 nM, or 1.0e+5 nM).

### Production of Targeted Therapeutics

Targeted therapeutics according to the present invention may be produced via various methods known in the art. In some embodiments, a targeted therapeutic is a fusion protein comprising a coupling moiety and a therapeutic protein (e.g., a lysosomal enzyme). It is contemplated in accordance with the invention, that the targeted therapeutic may be produced recombinantly. For example, a fusion protein according to the invention may be engineered using standard recombinant technology and produced using a cell culture system.

Various prokaryotic and eukaryotic cells may be used for producing fusion proteins including, without limitation, cell lines derived from bacteria strains, yeast strains, insect cells, animal cells, mammalian cells and human cells. Aspects of the present invention also provide for expression constructs and the generation of recombinant stable cell lines useful for expressing fusion proteins which are disclosed in the present specification. In addition, aspects of the present invention also provide methods for producing cell lines that express fusion proteins using nucleic acid sequences encoding the fusion proteins of the present specification.

### Nucleic Acids Encoding Recombinant Fusion Proteins

In some embodiments, nucleic acid molecules are provided comprising nucleic acid sequences encoding for a recombinant fusion protein (herein referred to as a transgene), such as GAA and Naglu fusion proteins described in various embodiments herein. In some embodiments, the nucleic acid encoding a transgene may be modified to provide increased expression of the fusion protein, which is also referred to as codon optimization. For example, the nucleic acid encoding a transgene can be modified by altering the open reading frame for the coding sequence. As used herein, the term “open reading frame” is synonymous with “ORF” and means any nucleotide sequence that is potentially able to encode a protein, or a portion of a protein. An open reading frame usually begins with a start codon (represented as, e.g. AUG for an RNA molecule and ATG in a DNA molecule in the standard code) and is read in codon-triplets until the frame ends with a STOP codon (represented as, e.g. UAA, UGA or UAG for an RNA molecule and TAA, TGA or TAG in a DNA molecule in the standard code). As used herein, the term “codon” means a sequence of three nucleotides in a nucleic acid molecule that specifies a particular amino acid during protein synthesis; also called a triplet or codon-triplet. For example, of the 64 possible codons in the standard genetic code, two codons, GAA and GAG encode the amino acid Glutamine whereas the codons AAA and AAG specify the amino acid Lysine. In the standard genetic code three codons are stop codons, which do not specify an amino acid. As used herein, the term “synonymous codon” means any and all of the codons that code for a single amino acid. Except for Methionine and Tryptophan, amino acids are coded by two to six synonymous codons. For example, in the standard genetic code the four synonymous codons that code for the amino acid Alanine are GCA, GCC, GCG and GCU, the two synonymous codons that specify Glutamine are GAA and GAG and the two synonymous codons that encode Lysine are AAA and AAG.

In some embodiments, a nucleic acid encoding the open reading frame of fusion protein may be modified using standard codon optimization methods. Various commercial algorithms for codon optimization are available and can be used to practice the present invention. Typically, codon optimization does not alter the encoded amino acid sequences. In some embodiments, codon optimization may lead to amino acids alteration such as substitution, deletion or insertion. Typically, such amino acid alteration does not substantially alter the protein activity.

In some embodiments, a nucleotide change may alter a synonymous codon within the open reading frame in order to agree with the endogenous codon usage found in a particular heterologous cell selected for expression. Alternatively or additionally, a nucleotide change may alter the G+C content within the open reading frame to better match the average G+C content of open reading frames found in endogenous nucleic acid sequence present in the heterologous host cell. A nucleotide change may also alter a polymononucleotide region or an internal regulatory or structural site found within a protein sequence. Thus, a variety of modified or optimized nucleotide sequences are envisioned including, without limitation, nucleic acid sequences providing increased expression of a fusion protein in a prokaryotic cell; yeast cell; insect cell; and in a mammalian cell.

### Expression Vectors

A nucleic acid sequence encoding a fusion protein as described in the present application, can be molecularly cloned (inserted) into a suitable vector for propagation or expression in a host cell. A wide variety of expression vectors can be used to practice the present invention, including, without limitation, a prokaryotic expression vector; a yeast expression vector; an insect expression vector and a mammalian expression vector. Exemplary vectors suitable for the present invention include, but are not limited to, viral based vectors (e.g., AAV based vectors, retrovirus based vectors, plasmid based vectors). Typically, a nucleic acid encoding a fusion protein is operably linked to various regulatory sequences or elements.

### Regulatory Sequences or Elements

Various regulatory sequences or elements may be incorporated in an expression vector suitable for the present invention. Exemplary regulatory sequences or elements include, but are not limited to, promoters, enhancers, repressors or suppressors, 5′ untranslated (or non-coding) sequences, introns, 3′ untranslated (or non-coding) sequences.

As used herein, a “Promoter” or “Promoter sequence” is a DNA regulatory region capable of binding an RNA polymerase in a cell (e.g., directly or through other promoter bound proteins or substances) and initiating transcription of a coding sequence. A promoter sequence is, in general, bound at its 3′ terminus by the transcription initiation site and extends upstream (5′ direction) to include the minimum number of bases or elements necessary to initiate transcription at any level. The promoter may be operably associated with or operably linked to the expression control sequences, including enhancer and repressor sequences or with a nucleic acid to be expressed. In some embodiments, the promoter may be inducible. In some embodiments, the inducible promoter may be unidirectional or bio-directional. In some embodiments, the promoter may be a constitutive promoter. In some embodiments, the promoter can be a hybrid promoter, in which the sequence containing the transcriptional regulatory region is obtained from one source and the sequence containing the transcription initiation region is obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N.Y., 1989, which is incorporated herein by reference). Commercial vectors suitable for inserting a transgene for expression in various host cells under a variety of growth and induction conditions are also well known in the art.

In some embodiments, a specific promoter may be used to control expression of the transgene in a mammalian host cell such as, but are not limited to, SRα-promoter (Takebe et al., Molec. and Cell. Bio. 8:466-472 (1988)), the human CMV immediate early promoter (Boshart et al., Cell 41:521-530 (1985); Foecking et al., Gene 45:101-105 (1986)), human CMV promoter, the human CMV5 promoter, the murine CMV immediate early promoter, the EF1-α-promoter, a hybrid CMV promoter for liver specific expression (e.g., made by conjugating CMV immediate early promoter with the transcriptional promoter elements of either human α-1-antitrypsin (HAT) or albumin (HAL) promoter), or promoters for hepatoma specific expression (e.g., wherein the transcriptional promoter elements of either human albumin (HAL; about 1000 bp) or human α-1-antitrypsin (HAT, about 2000 bp) are combined with a 145 long enhancer element of human α-1-microglobulin and bikunin precursor gene (AMBP); HAL-AMBP and HAT-AMBP); the SV40 early promoter region (Benoist at al., Nature 290:304-310 (1981)), the Orgyia pseudotsugata immediate early promoter, the herpes thymidine kinase promoter (Wagner at al., Proc. Natl. Acad. Sci. USA 78:1441-1445 (1981)); or the regulatory sequences of the metallothionein gene (Brinster et al., Nature 296:39-42 (1982)). In some embodiments, the mammalian promoter is a is a constitutive promoter such as, but not limited to, the hypoxanthine phosphoribosyl transferase (HPTR) promoter, the adenosine deaminase promoter, the pyruvate kinase promoter, the beta-actin promoter as well as other constitutive promoters known to those of ordinary skill in the art.

In some embodiments, a specific promoter may be used to control expression of a transgene in a prokaryotic host cell such as, but are not limited to, the β-lactamase promoter (Villa-Komaroff et al., Proc. Natl. Acad. Sci. USA 75:3727-3731 (1978)); the tac promoter (DeBoer et al., Proc. Natl. Acad. Sci. USA 80:21-25 (1983)); the T7 promoter, the T3 promoter, the M13 promoter or the M16 promoter; in a yeast host cell such as, but are not limited to, the GAL1, GAL4 or GAL10 promoter, the ADH (alcohol dehydrogenase) promoter, PGK (phosphoglycerol kinase) promoter, alkaline phosphatase promoter, glyceraldehyde-3-phosphate dehydrogenase III (TDH3) promoter, glyceraldehyde-3-phosphate dehydrogenase II (TDH2) promoter, glyceraldehyde-3-phosphate dehydrogenase I (TDH1) promoter, pyruvate kinase (PYK), enolase (ENO), or triose phosphate isomerase (TPI).

In some embodiments, the promoter may be a viral promoter, many of which are able to regulate expression of a transgene in several host cell types, including mammalian cells. Viral promoters that have been shown to drive constitutive expression of coding sequences in eukaryotic cells include, for example, simian virus promoters, herpes simplex virus promoters, papilloma virus promoters, adenovirus promoters, human immunodeficiency virus (HIV) promoters, Rous sarcoma virus promoters, cytomegalovirus (CMV) promoters, the long terminal repeats (LTRs) of Moloney murine leukemia virus and other retroviruses, the thymidine kinase promoter of herpes simplex virus as well as other viral promoters known to those of ordinary skill in the art.

In some embodiments, the gene control elements of an expression vector may also include 5′ non-transcribing and 5′ non-translating sequences involved with the initiation of transcription and translation, respectively, such as a TATA box, capping sequence, CAAT sequence, Kozak sequence and the like. Enhancer elements can optionally be used to increase expression levels of a polypeptide or protein to be expressed. Examples of enhancer elements that have been shown to function in mammalian cells include the SV40 early gene enhancer, as described in Dijkema et al., EMBO J. (1985) 4: 761 and the enhancer/promoter derived from the long terminal repeat (LTR) of the Rous Sarcoma Virus (RSV), as described in Gorman et al., Proc. Natl. Acad. Sci. USA (1982b) 79:6777 and human cytomegalovirus, as described in Boshart et al., Cell (1985) 41:521. Genetic control elements of an expression vector will also include 3′ non-transcribing and 3′non-translating sequences involved with the termination of transcription and translation. Respectively, such as a poly polyadenylation (polyA) signal for stabilization and processing of the 3′ end of an mRNA transcribed from the promoter. Poly A signals included, for example, the rabbit beta globin polyA signal, bovine growth hormone polyA signal, chicken beta globin terminator/polyA signal, or SV40 late polyA region.

### Selectable Markers

Expression vectors will preferably but optionally include at least one selectable marker. In some embodiments, the selectable maker is a nucleic acid sequence encoding a resistance gene operably linked to one or more genetic regulatory elements, to bestow upon the host cell the ability to maintain viability when grown in the presence of a cyctotoxic chemical and/or drug. In some embodiments, a selectable agent may be used to maintain retention of the expression vector within the host cell. In some embodiments, the selectable agent is may be used to prevent modification (i.e. methylation) and/or silencing of the transgene sequence within the expression vector. In some embodiments, a selectable agent is used to maintain episomal expression of the vector within the host cell. In some embodiments, the selectable agent is used to promote stable integration of the transgene sequence into the host cell genome. In some embodiments, an agent and/or resistance gene may include, but is not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), zeomycin, mycophenolic acid, or glutamine synthetase (GS, U.S. Pat. Nos. 5,122,464; 5,770,359; 5,827,739) for eukaryotic host cell; tetracycline, ampicillin, kanamycin or chlorampenichol for a prokaryotic host cell; and URA3, LEU2, HIS3, LYS2, HIS4, ADE8, CUP1 or TRP1 for a yeast host cell.

Expression vectors may be transfected, transformed or transduced into a host cell. As used herein, the terms “transfection,” “transformation” and “transduction” all refer to the introduction of an exogenous nucleic acid sequence into a host cell. In some embodiments, expression vectors containing nucleic acid sequences encoding a fusion therapeutic glycoprotein is transfected, transformed or transduced into a host cell. In some embodiments, one or more expression vectors containing nucleic acid sequences encoding a fusion therapeutic glycoprotein are transfected, transformed or transduced into a host cell sequentially. For example, a vector encoding a first fusion therapeutic glycoprotein protein may be transfected, transformed or transduced into a host cell, followed by the transfection, transformation or transduction of a vector encoding a second fusion therapeutic glycoprotein, and vice versa. Examples of transformation, transfection and transduction methods, which are well known in the art, include liposome delivery, i.e., Lipofectamine™ (Gibco BRL) Method of Hawley-Nelson, Focus 15:73 (1193), electroporation, CaPO₄ delivery method of Graham and van der Erb, Virology, 52:456-457 (1978), DEAE-Dextran medicated delivery, microinjection, biolistic particle delivery, polybrene mediated delivery, cationic mediated lipid delivery, transduction, and viral infection, such as, e.g., retrovirus, lentivirus, adenovirus adeno-associated virus and Baculovirus (Insect cells). General aspects of cell host transformations have been described in the art, such as by Axel in U.S. Pat. No. 4,399,216; Sambrook, supra, Chapters 1-4 and 16-18; Ausubel, supra, chapters 1, 9, 13, 15, and 16. For various techniques for transforming mammalian cells, see Keown et al., Methods in Enzymology (1989), Keown et al., Methods in Enzymology, 185:527-537 (1990), and Mansour et al., Nature, 336:348-352 (1988).

Once introduced inside cells, expression vectors may be integrated stably in the genome or exist as extra-chromosomal constructs. Vectors may also be amplified and multiple copies may exist or be integrated in the genome. In some embodiments, cells of the invention may contain 1, 2, 3, 4, 5, 6, 7, 8, 9, 10, 15, 20 or more copies of nucleic acids encoding a fusion therapeutic glycoprotein. In some embodiments, cells of the invention may contain multiple copies (e.g., 2, 3, 4, 5, 6, 7, 8, 9, 10, 15, 20 or more) of nucleic acids encoding one or more fusion therapeutic glycoproteins.

### Mammalian Cell Lines

Any mammalian cell or cell type susceptible to cell culture, and to expression of polypeptides, may be utilized in accordance with the present invention as a host cell. Non-limiting examples of mammalian cells that may be used in accordance with the present invention include HT1080 cells (Rasheed S, Nelson-Rees W A, Toth E M, Arnstein P, Gardner M B. Characterization of a newly derived human sarcoma cell line (HT1080). Cancer 33:1027-1033, 1974), human embryonic kidney 293 cells (HEK293), HeLa cells; BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells +/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). In some embodiments, a suitable mammalian cell is not a endosomal acidification-deficient cell. In some embodiments of the present invention, a suitable host cell is a CHO cell.

Additionally, any number of commercially and non-commercially available hybridoma cell lines that express polypeptides or proteins may be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

### Non-Mammalian Cell Lines

Any non-mammalian derived cell or cell type susceptible to cell culture, and to expression of polypeptides, may be utilized in accordance with the present invention as a host cell. Non-limiting examples of non-mammalian host cells and cell lines that may be used in accordance with the present invention include cells and cell lines derived from Pichia pastoris, Pichia methanolica, Pichia angusta, Schizosacccharomyces pombe, Saccharomyces cerevisiae, and Yarrowia lipolytica for yeast; Sodoptera frugiperda, Trichoplusis ni, Drosophila melangoster and Manduca sexta for insects; and Escherichia coli, Salmonella typhimurium, Bacillus subtilis, Bacillus lichenifonnis, Bacteroides fragilis, Clostridia perfringens, Clostridia difficile for bacteria; and Xenopus Laevis from amphibian.

In other embodiments, transgenic nonhuman mammals have been shown to produce therapeutic glycoproteins (e.g., lysosomal enzymes) in their milk. Such transgenic nonhuman mammals may include mice, rabbits, goats, sheep, porcines or bovines. See U.S. Pat. Nos. 6,118,045 and 7,351,410, each of which are hereby incorporated by reference in their entirety.

Any and all methods suitable for producing recombinant protein can be used to produce therapeutic protein of the present invention.

### Pharmaceutical Compositions and Administration

The present invention further provides pharmaceutical compositions containing targeted therapeutics according to the present invention. Typically, suitable pharmaceutical compositions contain at least one pharmaceutically acceptable excipient and are formulated for administration to humans.

For example, pharmaceutical compositions provided herein may be provided in a sterile injectable form (e.g., a form that is suitable for intravenous, intramuscular, subcutaneous, or intrathecal injection). For example, in some embodiments, pharmaceutical compositions are provided in a liquid dosage form that is suitable for injection. In some embodiments, pharmaceutical compositions are provided as powders (e.g., lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). In some embodiments, pharmaceutical compositions comprise one or more preservatives. In some embodiments, pharmaceutical compositions comprise no preservative.

Compositions of the pharmaceutical compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. By way of example, the composition may comprise between 0.1% and 100% (w/w) active ingredient.

Pharmaceutical compositions of the present invention may additionally comprise a pharmaceutically acceptable excipient, which, as used herein, may be or comprise solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

Targeted therapeutics described herein (or a composition or medicament containing a targeted therapeutics described herein) can be administered by any appropriate route generally known in the art. In some embodiments, a targeted therapeutic or a pharmaceutical composition containing the same is administered systemically. Systemic administration may be intravenous, intramuscular, intradermal, by inhalation, transdermal (topical), intraocular, subcutaneous, oral and/or transmucosal. In some embodiments, a targeted therapeutics or a pharmaceutical composition containing the same is administered by intramuscular injection. In some embodiments, a targeted therapeutics or a pharmaceutical composition containing the same is administered subcutaneously. Administration may be performed by injecting a composition into areas including, but not limited to, the thigh region, abdominal region, gluteal region, or scapular region. In some embodiments, a targeted therapeutics or a pharmaceutical composition containing the same is administered intravenously. More than one route can be used concurrently, if desired. All of the administration routes disclosed herein are generally known in the art, and the skilled artisan would know how to administer targeted therapeutics of the present invention by these routes.

In some embodiments, pharmaceutical compositions according to the present invention can be used for CNS delivery via various techniques and routes including, but not limited to, intraparenchymal, intracerebral, intravetricular cerebral (ICV), intrathecal (e.g., IT-Lumbar, IT-cisterna magna) administrations and any other techniques and routes for injection directly or indirectly to the CNS and/or CSF.

In some embodiments, pharmaceutical compositions according to the present invention can be used for intrathecal administration. As used herein, intrathecal administration (also referred to as intrathecal injection or intrathecal delivery) refers to an injection into the spinal canal (intrathecal space surrounding the spinal cord). Various formulations for intrathecal administration are described in WO/2011/163652, the contents of which are incorporated herein by reference.

According to the present invention, a pharmaceutical composition containing a targeted therapeutics may be injected at any region surrounding the spinal canal. In some embodiments, a pharmaceutical composition containing a targeted therapeutics is injected into the lumbar area or the cisterna magna or intraventricularly into a cerebral ventricle space. As used herein, the term “lumbar region” or “lumbar area” refers to the area between the third and fourth lumbar (lower back) vertebrae and, more inclusively, the L2-S1 region of the spine. Typically, intrathecal injection via the lumbar region or lumber area is also referred to as “lumbar IT delivery” or “lumbar IT administration.”

Various devices may be used for intrathecal delivery according to the present invention. In some embodiments, a device for intrathecal administration contains a fluid access port (e.g., injectable port); a hollow body (e.g., catheter) having a first flow orifice in fluid communication with the fluid access port and a second flow orifice configured for insertion into spinal cord; and a securing mechanism for securing the insertion of the hollow body in the spinal cord. As a non-limiting example, a suitable securing mechanism contains one or more nobs mounted on the surface of the hollow body and a sutured ring adjustable over the one or more nobs to prevent the hollow body (e.g., catheter) from slipping out of the spinal cord. In various embodiments, the fluid access port comprises a reservoir. In some embodiments, the fluid access port comprises a mechanical pump (e.g., an infusion pump). In some embodiments, an implanted catheter is connected to either a reservoir (e.g., for bolus delivery), or an infusion pump. The fluid access port may be implanted or external

In some embodiments, intrathecal administration may be performed by either lumbar puncture (i.e., slow bolus) or via a port-catheter delivery system (i.e., infusion or bolus). In some embodiments, the catheter is inserted between the laminae of the lumbar vertebrae and the tip is threaded up the thecal space to the desired level (generally L3-L4).

For injection, formulations of the invention can be formulated in liquid solutions. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

### Treatment of Pompe Disease, San B and Other Lysosomal Storage Diseases

The present invention may be used to effectively treat Pompe Disease, Sanfilippo Syndrome Type B and other lysosomal storage diseases.

Pompe disease, or Glycogen Storage Disease Type II, is an autosomal recessive metabolic disorder resulting from a deficiency or dysfunction of the lysosomal hydrolase acid alpha-glucosidase (GAA). GAA is localized to lysosomes and plays an important role in the catabolism of glycogen into glucose. In the absence of the enzyme, these glycogen accumulates within the cells, ultimately causing engorgement, followed by cellular death and tissue destruction. Due the widespread expression of the enzyme, multiple cells types and organ systems are affected in Pompe patients.

Unlike San B, which has CNS degeneration as the predominant defining clinical feature, Pompe disease is characterized by a degeneration within the peripheral tissues of the body. In particular, glycogen build-up with the body results in progressive muscle weakness (myopathy) through the body, specifically affecting the tissues of the heart, skeletal muscles, liver and kidneys. Typical findings are those of enlarged heart with non-specific conduction defects, along with several indicators of kidney disease, such as high levels of serum creatine kinase, aldolase, aspartate transaminase and lactic dehydrogenase. The disease typically manifests itself in the first several month of life, with cardiomegaly, hypotonia, cardiomyopathy, respiratory distress and muscle weakness. Some affected individuals experience a progressive loss of skeletal muscle, cardiac or kidney function, with most affected individuals dying of disease-associated complications in their first or second decade.

Sanfilippo Syndrome Type B (San B), or Mucopolysaccharidosis III B (MPS III B), is a heritable metabolic disorder resulting from a deficiency of the enzyme Naglu. Naglu is localized to lysosomes and plays an important role in the catabolism of glycosaminoglycans (GAGs) heparan- and dermatan-sulfate. In the absence of enzyme, these substrates accumulate within cells, ultimately causing engorgement, followed by cellular death and tissue destruction. Due to the widespread expression of enzyme, multiple cell types and organ systems are affected in MPS III B patients.

A defining clinical feature of San B is central nervous system (CNS) degeneration, which results in cognitive impairment (e.g., decrease in IQ). Additionally, MRI scans of affected individuals have revealed white matter lesions, dilated perivascular spaces in the brain parenchyma, ganglia, corpus callosum, and brainstem; atrophy; and ventriculomegaly (Wang et al. Molecular Genetics and Metabolism, 2009). The disease typically manifests itself in the first years of life with organomegaly and skeletal abnormalities. Some affected individuals experience a progressive loss of cognitive function, with most affected individuals dying of disease-associated complications in their first or second decade.

Compositions and methods of the present invention may be used to effectively treat individuals suffering from or susceptible to Pompe Disease or San B. The terms, “treat” or “treatment,” as used herein, refers to amelioration of one or more symptoms associated with the disease, prevention or delay of the onset of one or more symptoms of the disease, and/or lessening of the severity or frequency of one or more symptoms of the disease.

In some embodiments, treatment refers to partial or complete alleviation, amelioration, relief, inhibition, delay of onset, reduction of severity and/or incidence of impairment in a Pompe Disease or San B patient. As used herein, the term “impairment” includes various symptoms in various organ systems commonly associated with Pompe Disease and San B (e.g., in the brain and spinal cord or skeletal or heart muscle). Symptoms of neurological impairment may include, for example, e.g., cognitive impairment; white matter lesions; dilated perivascular spaces in the brain parenchyma, ganglia, corpus callosum, and/or brainstem; atrophy; and/or ventriculomegaly, among others. Symptoms often associated with Pompe Disease include, for example, weakness of skeletal muscle and heart failure and respiratory weakness.

The terms, “improve,” “increase” or “reduce,” as used herein, indicate values that are relative to a control. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with a lysosomal storage disease (e.g., San B, Pompe Disease), who is about the same age and/or gender as the individual suffering from the same lysosmal storage disease, who is being treated (to ensure that the stages of the disease in the treated individual and the control individual(s) are comparable).

The individual (also referred to as “patient” or “subject”) being treated is an individual (fetus, infant, child, adolescent, or adult human) having a lysosomal storage disease or having the potential to develop a lysosmal storage disease. In some embodiments, the lysosmal storage disease is Pompe Disease or Sanfilippo Syndrome. In some specific embodiments the lysosomal storage disease is Pompe Disease. The individual can have residual endogenous GAA or Naglu expression and/or activity, or no measurable activity. For example, the individual having Pompe Disease may have GAA expression levels that are less than about 30-50%, less than about 25-30%, less than about 20-25%, less than about 15-20%, less than about 10-15%, less than about 5-10%, less than about 0.1-5% of normal GAA expression levels. For example, the individual having San B may have Naglu expression levels that are less than about 30-50%, less than about 25-30%, less than about 20-25%, less than about 15-20%, less than about 10-15%, less than about 5-10%, less than about 0.1-5% of normal Naglu expression levels.

In some embodiments, the individual is an individual who has been recently diagnosed with the disease. Typically, early treatment (treatment commencing as soon as possible after diagnosis) is important to minimize the effects of the disease and to maximize the benefits of treatment.

All literature and patent and patent publication citations herein are incorporated herein by reference in their entirety.

The invention will be more fully understood by reference to the following examples. They should not, however, be construed as limiting the scope of the invention.

### Examples

### Example 1: Generation of Components for PCSK9 Fusion Proteins

The present invention provides, among other things, methods and compositions for lysosomal targeting of a targeted therapeutic (e.g., a coupling moiety fused to a lysosmal enzyme) based on formation of a lysosomal delivery complex. The current example, demonstrates a general method for producing one or more targeted therapeutics, by generating a translational fusion protein between a lysosmal enzyme and a coupling moiety.

The lysosomal enzymes acid alpha-glucosidase (GAA) and N-Acetylglucosaminidase (Naglu) were chosen as a candidate proteins, since it has been demonstrated that deficiency of each individual protein plays a central role in the development of Pompe disease and Sanpfilippo Syndrome (Mucopolysaccharidosis III) Type B, respectively. However, it will be understood by one skilled in the art, that such an approach is broadly applicable in generating fusion therapeutics for conditions associated with any lysosomal storage disease. It is contemplated that suitable fusion therapeutics of the current invention facilitate cellular uptake and lysosomal targeting and have an enzyme activity substantially similar to the native enzyme.

Coupling moieties may be associated with suitable therapeutic enzymes (e.g., lysosomal enzymes) covalently or non-covalently. For example, a coupling moiety may be chemically conjugated to a therapeutic enzyme. Alternatively, a coupling moiety may be fused to a therapeutic enzyme, creating a fusion protein. In this example, a series of two constructs were created, each designed to express GAA or Naglu, fused to a coupling moiety.

### GAA Targeted Therapeutic

An exemplary GAA fusion protein is created by connecting a nucleid acid encoding a heavy chain of an anti-PCSK9 monoclonal human antibody (which may block binding between PSCK9 and LDLR) to a nucleic acid encoding GAA via an intervening GGG-encoding linker. The amino acid sequence resulting from the ranslation of such a nucleic acid is shown below (SEQ ID NO:11).

(SEQ ID NO: 11)
MEFGLSWLFLVAILKGVQCQVQLVQSGAEVKKPGASVKVSCKASGYTFTS
YYMHWVRQAPGQGLEWMGEISPFGGRTNYNEKFKSRVTMTRDTSTSTVYM
ELSSLRSEDTAVYYCARERPLYASDLWGQGTTVTVSSASTKGPSVFPLAP
SSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPP
VAGPSVFLFPPKPKDTLMISRTPEVTWVVVDVSHEDPEVQFNWYVDGVEV
HNAKTKPREEQFNSTFCVVSVLTVVHQDWLNGKEYKCKVSNKGLPAPIEK
TISKTKGQPREPQVYTLPPSREEMTKNQVSLTCLVKGFYPSDIAVEWESN
GQPENNYKTTPPMLDSDGSFFLYSKLTVDKSRWQQGNVFSCSVMHEALHN
HYTQKSLSLSPGKGGGAHPGRPRAVPTQCDVPPNSRFDCAPDKAITQEQC
EARGCCYIPAKQGLQGAQMGQPWCFFPPSYPSYKLENLSSSEMGYTATLT
RTTPTFFPKDILTLRLDVMMETENRLHFTIKDPANRRYEVPLETPHVHSR
APSPLYSVEFSEEPFGVIVRRQLDGRVLLNTTVAPLFFADQFLQLSTSLP
SQYITGLAEHLSPLMLSTSWTRITLWNRDLAPTPGANLYGSHPFYLALED
GGSAHGVFLLNSNAMDVVLQPSPALSWRSTGGILDVYIFLGPEPKSVVQQ
YLDVVGYPFMPPYWGLGFHLCRWGYSSTAITRQVVENMTRAHFPLDVQWN
DLDYMDSRRDFTFNKDGFRDFPAMVQELHQGGRRYMMIVDPAISSSGPAG
SYRPYDEGLRRGVFITNETGQPLIGKVWPGSTAFPDFTNPTALAWWEDMV
AEFHDQVPFDGMWIDMNEPSNFIRGSEDGCPNNELENPPYVPGVVGGTLQ
AATICASSHQFLSTHYNLHNLYGLTEAIASHRALVKARGTRPFVISRSTF
AGHGRYAGHWTGDVWSSWEQLASSVPEILQFNLLGVPLVGADVCGFLGNT
SEELCVRWTQLGAFYPFMRNHNSLLSLPQEPYSFSEPAQQAMRKALTLRY
ALLPHLYTLFHQAHVAGETVARPLFLEFPKDSSTWTVDHQLLWGEALLIT
PVLQAGKAEVTGYFPLGTWYDLQTVPVEALGSLPPPPAAPREPAIHSEGQ
WVTLPAPLDTINVHLRAGYIIPLQGPGLTTTESRQQPMALAVALTKGGEA
RGELFWDDGESLEVLERGAYTQVIFLARNNTIVNELVRVTSEGAGLQLQK
VTVLGVATAPQQVLSNGVPVSNFTYSPDTKVLDICVSLLMGEQFLVSWC

1.) Anti-PCSK9 human monoclonal antibody (J16) heavy chain

Signal peptide of IgG heavy chain—in bold

Variable region of J16 IgG heavy chain—italics

Constant region of heavy chain (IgG2)—underlined

2.) GGG Linker—in bold
3.) human Mature Form GAA Protein—in italics

Alternatively, an exemplary GAA fusion protein is created by connecting a nucleid acid encoding a single-chain scFv molecule of an anti-PCSK9 monoclonal human antibody (which blocks binding between PSCK9 and LDLR) to a nucleic acid encoding GAA via an intervening GGG-encoding linker. The amino acid sequence resulting from the translation of such a nucleic acid is shown below (SEQ ID NO:12).

(SEQ ID NO: 12)
MEFGLSWLFLVAILKGVQCQVQLVQSGAEVKKPGASVKVSCKASGYTFTS
YYMHWVRQAPGQGLEWMGEISPFGGRTNYNEKFKSRVTMTRDTSTSTVYM
ELSSLRSEDTAVYYCARERPLYASDLWGQGTTVTVSSGGGGSGGGGSGGG
GSDIQMTQSPSSLSASVGDRVTITCRASQGISSALAWYQQKPGKAPKLLI
YSASYRYTGVPSRFSGSGSGTDFTFTISSLQPEDIATYYCQQRYSLWRTF
GQGTKLEIKRGGGAHPGRPRAVPTQCDVPPNSRFDCAPDKAITQEQCEAR
GCCYIPAKQGLQGAQMGQPWCFFPPSYPSYKLENLSSSEMGYTATLTRTT
PTFFPKDILTLRLDVMMETENRLHFTIKDPANRRYEVPLETPHVHSRAPS
PLYSVEFSEEPFGVIVRRQLDGRVLLNTTVAPLFFADQFLQLSTSLPSQY
ITGLAEHLSPLMLSTSWTRITLWNRDLAPTPGANLYGSHPFYLALEDGGS
AHGVFLLNSNAMDVVLQPSPALSWRSTGGILDVYIFLGPEPKSVVQQYLD
VVGYPFMPPYWGLGFHLCRWGYSSTAITRQVVENMTRAHFPLDVQWNDLD
YMDSRRDFTFNKDGFRDFPAMVQELHQGGRRYMMIVDPAISSSGPAGSYR
PYDEGLRRGVFITNETGQPLIGKVWPGSTAFPDFTNPTALAWWEDMVAEF
HDQVPFDGMWIDMNEPSNFIRGSEDGCPNNELENPPYVPGVVGGTLQAAT
ICASSHQFLSTHYNLHNLYGLTEAIASHRALVKARGTRPFVISRSTFAGH
GRYAGHWTGDVWSSWEQLASSVPEILQFNLLGVPLVGADVCGFLGNTSEE
LCVRWTQLGAFYPFMRNHNSLLSLPQEPYSFSEPAQQAMRKALTLRYALL
PHLYTLFHQAHVAGETVARPLFLEFPKDSSTWTVDHQLLWGEALLITPVL
QAGKAEVTGYFPLGTWYDLQTVPVEALGSLPPPPAAPREPAIHSEGQWVT
LPAPLDTINVHLRAGYIIPLQGPGLTTTESRQQPMALAVALTKGGEARGE
LFWDDGESLEVLERGAYTQVIFLARNNTIVNELVRVTSEGAGLQLQKVTV
LGVATAPQQVLSNGVPVSNFTYSPDTKVLDICVSLLMGEQFLVSWC

1.) Anti-PCSK9 human monoclonal antibody (J16) scFv

Signal peptide of IgG heavy chain—in bold

Variable region of J16 IgG heavy chain—italics

GGGGS3 Linker—in bold

Variable region of J16 IgG light chain—underlined

2.) GGG Linker—in bold
3.) human Mature Form GAA Protein—in italics

### Naglu Targeted Therapeutic

An exemplary Naglu fusion protein is created by connecting a nucleid acid encoding a heavy chain of an anti-PCSK9 monoclonal human antibody (which blocks binding between PSCK9 and LDLR) to a nucleic acid encoding Naglu via an intervening GGG-encoding linker. The amino acid sequence resulting from the ranslation of such a nucleic acid is shown below (SEQ ID NO:13).

(SEQ ID NO: 13)
MEFGLSWLFLVAILKGVQCQVQLVQSGAEVKKPGASVKVSCKASGYTFTS
YYMHWVRQAPGQGLEWMGEISPFGGRTNYNEKFKSRVTMTRDTSTSTVYM
ELSSLRSEDTAVYYCARERPLYASDLWGQGTTVTVSSASTKGPSVFPLAP
SSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPP
VAGPSVFLFPPKPKDTLMISRTPEVTWVVVDVSHEDPEVQFNWYVDGVEV
HNAKTKPREEQFNSTFCVVSVLTVVHQDWLNGKEYKCKVSNKGLPAPIEK
TISKTKGQPREPQVYTLPPSREEMTKNQVSLTCLVKGFYPSDIAVEWESN
GQPENNYKTTPPMLDSDGSFFLYSKLTVDKSRWQQGNVFSCSVMHEALHN
HYTQKSLSLSPGKGGGDEAREAAAVRALVARLLGPGPAADFSVSVERALA
AKPGLDTYSLGGGGAARVRVRGSTGVAAAAGLHRYLRDFCGCHVAWSGSQ
LRLPRPLPAVPGELTEATPNRYRYYQNVCTQSYSFVWWDWARWEREIDWM
ALNGINLALAWSGQEAIWQRVYLALGLTQAEINEFFTGPAFLAWGRMGNL
HTWDGPLPPSWHIKQLYLQHRVLDQMRSFGMTPVLPAFAGHVPEAVTRVF
PQVNVTKMGSWGHFNCSYSCSFLLAPEDPIFPIIGSLFLRELIKEFGTDH
IYGADTFNEMQPPSSEPSYLAAATTAVYEAMTAVDTEAVWLLQGWLFQHQ
PQFWGPAQIRAVLGAVPRGRLLVLDLFAESQPVYTRTASFQGQPFIWCML
HNFGGNHGLFGALEAVNGGPEAARLFPNSTMVGTGMAPEGISQNEVVYSL
MAELGWRKDPVPDLAAWVTSFAARRYGVSHPDAGAAWRLLLRSVYNCSGE
ACRGHNRSPLVRRPSLQMNTSIWYNRSDVFEAWRLLLTSAPSLATSPAFR
YDLLDLTRQAVQELVSLYYEEARSAYLSKELASLLRAGGVLAYELLPALD
EVLASDSRFLLGSWLEQARAAAVSEAEADFYEQNSRYQLTLWGPEGNILD
YANKQLAGLVANYYTPRWRLFLEALVDSVAQGIPFQQHQFDKNVFQLEQA
FVLSKQRYPSQPRGDTVDLAKKIFLKYYPRWVAGSW_

1.) Anti-PCSK9 human monoclonal antibody (J16) heavy chain

Signal peptide of IgG heavy chain—in bold

Variable region of J16 IgG heavy chain—italics

Constant region of heavy chain (IgG2)—underlined

2.) GGG Linker—in bold
3.) human Mature Form of Naglu—in italics

Alternatively, an exemplary Naglu fusion protein is created by connecting a nucleid acid encoding a single-chain scFv molecule of an anti-PCSK9 monoclonal human antibody (which blocks binding between PSCK9 and LDLR) to a nucleic acid encoding Naglu via an intervening GGG-encoding linker. The amino acid sequence resulting from the ranslation of such a nucleic acid is shown below (SEQ ID NO:14).

(SEQ ID NO: 14)
MEFGLSWLFLVAILKGVQCQVQLVQSGAEVKKPGASVKVSCKASGYTFTS
YYMHWVRQAPGQGLEWMGEISPFGGRTNYNEKFKSRVTMTRDTSTSTVYM
ELSSLRSEDTAVYYCARERPLYASDLWGQGTTVTVSSGGGGSGGGGSGGG
GSDIQMTQSPSSLSASVGDRVTITCRASQGISSALAWYQQKPGKAPKLLI
YSASYRYTGVPSRFSGSGSGTDFTFTISSLQPEDIATYYCQQRYSLWRTF
GQGTKLEIKRGGGDEAREAAAVRALVARLLGPGPAADFSVSVERALAAKP
GLDTYSLGGGGAARVRVRGSTGVAAAAGLHRYLRDFCGCHVAWSGSQLRL
PRPLPAVPGELTEATPNRYRYYQNVCTQSYSFVWWDWARWEREIDWMALN
GINLALAWSGQEAIWQRVYLALGLTQAEINEFFTGPAFLAWGRMGNLHTW
DGPLPPSWHIKQLYLQHRVLDQMRSFGMTPVLPAFAGHVPEAVTRVFPQV
NVTICMGSWGHFNCSYSCSFLLAPEDPIFPIIGSLFLRELIKEFGTDHIY
GADTFNEMQPPSSEPSYLAAATTAVYEAMTAVDTEAVWLLQGWLFQHQPQ
FWGPAQIRAVLGAVPRGRLLVLDLFAESQPVYTRTASFQGQPFIWCMLHN
FGGNHGLFGALEAVNGGPEAARLFPNSTMVGTGMAPEGISQNEVVYSLMA
ELGWRKDPVPDLAAWVTSFAARRYGVSHPDAGAAWRLLLRSVYNCSGEAC
RGHNRSPLVRRPSLQMNTSIWYNRSDVFEAWRLLLTSAPSLATSPAFRYD
LLDLTRQAVQELVSLYYEEARSAYLSKELASLLRAGGVLAYELLPALDEV
LASDSRFLLGSWLEQARAAAVSEAEADFYEQNSRYQLTLWGPEGNILDYA
NKQLAGLVANYYTPRWRLFLEALVDSVAQGIPFQQHQFDKNVFQLEQAFV
LSKQRYPSQPRGDTVDLAKKIFLKYYPRWVAGSW

1.) Anti-PCSK9 human monoclonal antibody (J16) scFv

Signal peptide of IgG heavy chain—in bold

Variable region of J16 IgG heavy chain—italics

GGGGS3 Linker—in bold

Variable region of J16 IgG light chain—underlined

2.) GGG Linker—in bold
3.) human Mature Form of Naglu—in italics

Nucleic acids encoding a fusion protein (including those exemplified above) can be subcloned into mammalian expression vectors of choice. These expression constructs may then be transfected into a cell line (human or from other species), and the cell line may be screened to generate over-expressing cell clones. In one embodiment, cell clones overexpressing heavy chain fusion proteins (e.g., SEQ ID NO: 11 or 13) are also transfected with expression vectors expressing immunoglobulin light chains of choice (e.g., J16), using any one of the standard procedures known in the art. The overall result is a cell line that over-expresses antibodies or fragments thereof that are modified in accordance with the present invention.

Nucleic acids encoding fusion proteins according the present invention may encode precursor forms of a therapeutic enzyme (e.g., lysosomal enzyme), for example including a N-terminal signal- or pro-peptide.

For protein based assays and receptor binding experiments, recombinant protein may be produced in a wave bioreactor, using a mammalian cell culture expressing system (expressing the nucleic acids disclosed herein for example). Following expression, fusion proteins may be purified using conventional protein purification methods.

### Example 2: Activity Assay

Following purification, each fusion protein is evaluated for proper function, by examining its specific activity and enzyme kinetics using a well-defined cleavable substrate. Based on this analysis, GAA and Naglu therapeutic fusion protein binding constants and specificity for the enzyme substrate are compared to each respective wildtype lysosomal enzyme, to ensure enzyme function is similar to the native protein.

### Example 3: Fusion Protein Binding Studies

Studies will also be carried out to determine the binding properties of either antibody-GAA, scFv-GAA, antibody-Naglu or scFv-Naglu, and evaluate their specificity for PCSK9. For example, a surface plasmone resonance (SPR) assay will be employed using standard techniques. Towards that end, for example, PCSK9 serving as “ligand” is diluted in immobilization buffer and bound to the dextran surface of a SPR sensor chip housed in a microfluidic system. A solution containing purified fusion proteins, either antibody-GAA, scFv-GAA, antibody-Naglu or scFv-Naglu, serving as the “analyte”, is then injected into the device. Secondly, either the antibody-GAA, scFv-GAA, antibody-Naglu or scFv-Naglu, serving as “ligand,” is diluted in immobilization buffer and bound to the dextran surface of a SPR sensor chip housed in a microfluidic system. A solution containing PCSK9, serving as the “analyte,” is then injected into the device. Thirdly, a “capturing molecule,” such as anti-GAA antibody or anti-Naglu antibody, is diluted in immobilization buffer and bound to the dextran surface of a SPR sensor chip housed in a microfluidic system. Next, a solution containing either antibody-GAA or scFv-GAA, or antibody-Naglu or scFv-Naglu, depending on the capturing molecule used, serving as the “ligand,” is injected into the microflow system and run over the surface to bind the antibody to form a “capture complex.” A solution containing PCSK9, serving as the “analyte,” is then injected into the device.

In all three approaches, as the solution runs over the SPR sensor chip, the analyte binds to the ligant and/or capture complex, and an increase in SPR signal (expressed in response units, RU) is observed. After a predetermined period of time, a solution without the analyte is injected into the microfluidic device, resulting in dissociation of the interaction between analyte and ligant and/or capture complex, and thus a decrease in SPR signal.

Experimental SPR assay conditions where PCSK9 serves as the ligand are described in more detail in Table 4 below.

TABLE 4
Experimental Design For Exemplary
Surface Plasmone Resonance Assay

| | | | | | Associ- | Dissoci- |
|---|---|---|---|---|---|---|
| | | Analyte | | Flow | ation | ation |
| Ligand | Analyte | Conc. | | Rate | Time | Time |
| PCSK9 | Antibody-GAA | 0 | nM | 30 μl/min | 300 sec | 300 sec |
| | Antibody-GAA | 0.625 | nM | | | |
| | Antibody-GAA | 1.25 | nM | | | |
| | Antibody-GAA | 2.5 | nM | | | |
| | Antibody-GAA | 5 | nM | | | |
| | Antibody-GAA | 10 | nM | | | |
| | Antibody-GAA | 20 | nM | | | |
| PCSK9 | scFv-GAA | 0 | nM | 30 μl/min | 300 sec | 300 sec |
| | scFv-GAA | 0.625 | nM | | | |
| | scFv-GAA | 1.25 | nM | | | |
| | scFv-GAA | 2.5 | nM | | | |
| | scFv-GAA | 5 | nM | | | |
| | scFv-GAA | 10 | nM | | | |
| | scFv-GAA | 20 | nM | | | |
| PCSK9 | Antibody- Naglu | 0 | nM | 30 μl/min | 300 sec | 300 sec |
| | Antibody- Naglu | 0.625 | nM | | | |
| | Antibody- Naglu | 1.25 | nM | | | |
| | Antibody- Naglu | 2.5 | nM | | | |
| | Antibody- Naglu | 5 | nM | | | |
| | Antibody- Naglu | 10 | nM | | | |
| | Antibody- Naglu | 20 | nM | | | |
| PCSK9 | scFv- Naglu | 0 | nM | 30 μl/min | 300 sec | 300 sec |
| | scFv- Naglu | 0.625 | nM | | | |
| | scFv- Naglu | 1.25 | nM | | | |
| | scFv- Naglu | 2.5 | nM | | | |
| | scFv- Naglu | 5 | nM | | | |
| | scFv- Naglu | 10 | nM | | | |
| | scFv- Naglu | 20 | nM | | | |

To evaluate the overall specificity of each fusion protein, a competitive inhibition study using a SPR assay is also be performed. PCSK9 in solution (co-injected with analyte) is used as an “inhibitor protein.” Briefly, PCSK9 is diluted in immobilization buffer and bound to the dextran surface of a SPR sensor chip housed in a microfluidic system. Next, a solution containing fusion proteins (antibody-GAA, scFv-GAA, antibody-Naglu or scFv-Naglu) with or without 20 μM PCSK9 is injected into the device and analyzed for binding. After a predetermined period of time, a solution without the analyte is injected into the microfluidic device, dissociating any possible interaction between the analyte and the ligant, and resulting in a decrease in SPR signal. The experimental conditions used for the assay are described on more detail in Table 5 below.

TABLE 5
Experimental Design For Exemplary Surface Plasmone Resonance Assay

| | | | | Inhibitor | | | |
|---|---|---|---|---|---|---|---|
| | | Analyte | | PCSK9 | Flow | Association | Dissociation |
| Ligand | Analyte | Conc. | | (Conc.) | Rate | Time | Time |
| PCSK9 | Antibody-GAA | 20 | nM | 0.0 μM | 30 μl/min | 300 sec | 300 sec |
| | Antibody-GAA | 0 | nM | 20 μM | | | |
| | Antibody-GAA | 0.625 | nM | 20 μM | | | |
| | Antibody-GAA | 1.25 | nM | 20 μM | | | |
| | Antibody-GAA | 2.5 | nM | 20 μM | | | |
| | Antibody-GAA | 5 | nM | 20 μM | | | |
| | Antibody-GAA | 10 | nM | 20 μM | | | |
| | Antibody-GAA | 20 | nM | 20 μM | | | |
| PCSK9 | scFv-GAA | 20 | nM | 0.0 μM | 30 μl/min | 300 sec | 300 sec |
| | scFv-GAA | 0 | nM | 20 μM | | | |
| | scFv-GAA | 0.625 | nM | 20 μM | | | |
| | scFv-GAA | 1.25 | nM | 20 μM | | | |
| | scFv-GAA | 2.5 | nM | 20 μM | | | |
| | scFv-GAA | 5 | nM | 20 μM | | | |
| | scFv-GAA | 10 | nM | 20 μM | | | |
| | scFv-GAA | 20 | nM | 20 μM | | | |
| PCSK9 | Antibody-Naglu | 20 | nM | 0.0 μM | 30 μl/min | 300 sec | 300 sec |
| | Antibody-Naglu | 0 | nM | 20 μM | | | |
| | Antibody-Naglu | 0.625 | nM | 20 μM | | | |
| | Antibody-Naglu | 1.25 | nM | 20 μM | | | |
| | Antibody-Naglu | 2.5 | nM | 20 μM | | | |
| | Antibody-Naglu | 5 | nM | 20 μM | | | |
| | Antibody-Naglu | 10 | nM | 20 μM | | | |
| | Antibody-Naglu | 20 | nM | 20 μM | | | |
| PCSK9 | scFv-Naglu | 20 | nM | 0.0 μM | 30 μl/min | 300 sec | 300 sec |
| | scFv-Naglu | 0 | nM | 20 μM | | | |
| | scFv-Naglu | 0.625 | nM | 20 μM | | | |
| | scFv-Naglu | 1.25 | nM | 20 μM | | | |
| | scFv-Naglu | 2.5 | nM | 20 μM | | | |
| | scFv-Naglu | 5 | nM | 20 μM | | | |
| | scFv-Naglu | 10 | nM | 20 μM | | | |
| | scFv-Naglu | 20 | nM | 20 μM | | | |

A SPR competition study is also performed where the concentration of each fusion protein is held constant and assayed against varying concentrations of inhibitor protein PCSK9. Briefly, PCSK9, the “capturing molecule” is diluted in immobilization buffer and bound on the dextran surface of a SPR sensor chip housed in a microfluidic system. A solution containing each purified fusion protein at 20 nM, along with 0-1.5 uM of PCSK9, is injected into the device and analyzed for binding. After a predetermined period of time, a solution without the analyte is injected into the microfluidic device, dissociating any possible interaction between the analyte and the ligand, and resulting in a decrease in SPR signal. The experimental conditions for use in performing the assay are described in more detail in Table 6 below.

TABLE 6
Experimental Design For Exemplary Surface Plasmone Resonance Assay

| | | | Inhibitor | | | | |
|---|---|---|---|---|---|---|---|
| | | Analyte | PCSK9 | | Flow | Association | Dissociation |
| Ligand | Analyte | Conc. | (Conc.) | | Rate | Time | Time |
| PCSK9 | Antibody-GAA | 20 nM | 0.0 | nM | 30 μl/min | 300 sec | 300 sec |
| | Antibody-GAA | 20 nM | 25 | nM | | | |
| | Antibody-GAA | 20 nM | 50 | nM | | | |
| | Antibody-GAA | 20 nM | 100 | nM | | | |
| | Antibody-GAA | 20 nM | 200 | nM | | | |
| | Antibody-GAA | 20 nM | 400 | nM | | | |
| | Antibody-GAA | 20 nM | 600 | nM | | | |
| | Antibody-GAA | 20 nM | 1.0 | μM | | | |
| | Antibody-GAA | 20 nM | 1.5 | μM | | | |
| PCSK9 | scFv-GAA | 20 nM | 0.0 | nM | 30 μl/min | 300 sec | 300 sec |
| | scFv-GAA | 20 nM | 25 | nM | | | |
| | scFv-GAA | 20 nM | 50 | nM | | | |
| | scFv-GAA | 20 nM | 100 | nM | | | |
| | scFv-GAA | 20 nM | 200 | nM | | | |
| | scFv-GAA | 20 nM | 400 | nM | | | |
| | scFv-GAA | 20 nM | 600 | nM | | | |
| | scFv-GAA | 20 nM | 1.0 | μM | | | |
| | scFv-GAA | 20 nM | 1.5 | μM | | | |
| PCSK9 | Antibody-Naglu | 20 nM | 0.0 | nM | 30 μl/min | 300 sec | 300 sec |
| | Antibody-Naglu | 20 nM | 25 | nM | | | |
| | Antibody-Naglu | 20 nM | 50 | nM | | | |
| | Antibody-Naglu | 20 nM | 100 | nM | | | |
| | Antibody-Naglu | 20 nM | 200 | nM | | | |
| | Antibody-Naglu | 20 nM | 400 | nM | | | |
| | Antibody-Naglu | 20 nM | 600 | nM | | | |
| | Antibody-Naglu | 20 nM | 1.0 | μM | | | |
| | Antibody-Naglu | 20 nM | 1.5 | μM | | | |
| PCSK9 | scFv-Naglu | 20 nM | 0.0 | nM | 30 μl/min | 300 sec | 300 sec |
| | scFv-Naglu | 20 nM | 25 | nM | | | |
| | scFv-Naglu | 20 nM | 50 | nM | | | |
| | scFv-Naglu | 20 nM | 100 | nM | | | |
| | scFv-Naglu | 20 nM | 200 | nM | | | |
| | scFv-Naglu | 20 nM | 400 | nM | | | |
| | scFv-Naglu | 20 nM | 600 | nM | | | |
| | scFv-Naglu | 20 nM | 1.0 | μM | | | |
| | scFv-Naglu | 20 nM | 1.5 | μM | | | |

### Example 4: In Vitro Studies

### Cellular Uptake Assays

Studies may also be performed to assess lysosomal targeting and cellular uptake of lysosomal targeted therapeutics, in accordance with the claimed invention. In this particular representative example, a lysosmal targeting assay is utilized that uses PCSK9 in complex with one of the fusion proteins, either antibody-GAA, scFv-GAA, antibody-Naglu or scFv-Naglu. However, one skilled in the art will appreciate that Example 4 teaches a general assay method that may be used to evaluate any lysosomal targeted therapeutic in accordance with the teachings of the instant application. The cell line of choice for this assay is the mouse myoblast cell line C2C12 cell (Yaffe D. and Saxel O; Serial passaging and differentiation of myogenic cells isolated from dystrophic mouse muscle; Nature 270 (5639): 725-727 (1977)). C2C12 cells are grown to confluence and treated with a solution of PCSK9 in complex with one of the fusion proteins, either antibody-GAA, scFv-GAA, antibody-Naglu or scFv-Naglu. After a specified period of time, supernatant is removed, cells washed repeatedly; and following lysis each sample is assayed for Naglu and/or GAA enzyme activity.

Visualization of Lysosomal Targeting and Entry of Either Antibody-GAA, scFv-GAA, Antibody-NAGLU or scFv-NAGLU

Studies are also be carried out to evaluate cellular lysosomal targeting and entry using fluorescent immunomicroscopy. For the study, C2C12 cells are treated with or without recombinant PCSK9 in complex with one of the fusion proteins, either antibody-GAA, scFv-GAA, antibody-Naglu or scFv-Naglu. Following treatment, the cells are fixed and prepared for staining. Both control and treated cells are stained using antibodies specific for each lysosomal protein (GAA or Naglu) along with Lamp-1, a lysosome specific protein biomarker. Cells are assayed for cellular internalization of each fusion protein by immunofluroescent microscopy.

We claim:

1. A targeted therapeutic comprising:
(i) a lysosomal enzyme; and
(ii) a coupling moiety that binds specifically to a proprotein convertase protein.

2. The targeted therapeutic of claim 1, wherein the proprotein convertase protein is selected from the group consisting of PC1/3; PC2; Furin; PC4; PC5/6; PACE4, PC7, SKI-1/S1P and PCSK9.

3. The targeted therapeutic of claim 2, wherein the proprotein convertase is PCSK9.

4. The targeted therapeutic of any one of the preceding claims, wherein the lysosomal enzyme is selected from Table 3.

5. The targeted therapeutic of claim 4, wherein the lysosomal enzyme is acid alpha-glycosidase (GAA).

6. The targeted therapeutic of claim 5, wherein the acid alpha-glycosidase comprises an amino acid sequence at least 80%, 90% or 95% identical to SEQ ID NO:1.

7. The targeted therapeutic of claim 5, wherein the acid alpha-glycosidase comprises an amino acid sequence identical to SEQ ID NO:1.

8. The targeted therapeutic of claim 4, wherein the lysosomal enzyme is alpha-N-acetyl-glucosaminidase (Naglu).

9. The targeted therapeutic of claim 8, wherein the alpha-N-acetyl-glucosaminidase comprises an amino acid sequence at least 80%, 90% or 95% identical to SEQ ID NO:4.

10. The targeted therapeutic of claim 8, wherein the alpha-N-acetyl-glucosaminidase comprises an amino acid sequence identical to SEQ ID NO:4.

11. The targeted therapeutic of any one of the preceding claims, wherein the coupling moiety is a peptide.

12. The targeted therapeutic of claim 11, wherein the coupling moiety is fused to the lysosomal enzyme creating a fusion protein.

13. The targeted therapeutic of claim 12, wherein the coupling moiety is fused to the N-terminus of the lysosomal enzyme.

14. The targeted therapeutic of claim 12, wherein the coupling moiety is fused to the C-terminus of the lysosomal enzyme.

15. The targeted therapeutic of any one of claims 11-14, wherein the targeted therapeutic further comprises a linker joining the lysosomal enzyme and the coupling moiety.

16. The targeted therapeutic of claim 15, wherein the linker is a peptide linker.

17. The targeted therapeutic of claim 16, wherein the peptide linker comprises a sequence of three glycine residues.

18. The targeted therapeutic of claim 16, wherein the peptide linker comprises a cleavage site.

19. The targeted therapeutic of claim 18, wherein the cleavage site comprises a lysosomal protease recognition site.

20. The targeted therapeutic of any one of the preceding claims, wherein the coupling moiety interferes with binding between the proprotein convertase protein and an LDL receptor.

21. The targeted therapeutic of claim 20, wherein binding between the proprotein convertase protein and the LDL receptor is reduced by at least 50%, 80%, 85%, 90% or 95%.

22. The targeted therapeutic of any one of the preceding claims, wherein binding of the coupling moiety to PCSK9 protein alters subsequent binding between the PCSK9 protein and one or more secondary binding proteins selected from the group consisting of Amyloid Precursor-like Protein 2 (APLP2), Dynamin, Amyloid Precursor Protein (APP), Autosomal Recessive Hypercholesterolemia (ARH) protein, Low Density Lipoprotein Receptor-related Protein 8 (Lrp8) and combinations thereof.

23. The targeted therapeutic of claim 22, wherein binding between the PCSK9 protein and the one or more secondary binding proteins is enhanced by at least 50%, 80%, 85%, 90% or 95%, compared to binding by PCSK9 alone.

24. The targeted therapeutic of any one of claims 11-23, wherein the coupling moiety is an antibody or antibody fragment.

25. The targeted therapeutic of claim 24, wherein the antibody is a monoclonal antibody.

26. The targeted therapeutic of claim 25, wherein the monoclonal antibody is selected from the group consisting of a human antibody, mouse antibody and a rabbit antibody.

27. The targeted therapeutic of claim 25, wherein the antibody is a humanized mouse antibody.

28. The targeted therapeutic of claim 25, wherein the antibody is a human antibody.

29. The targeted therapeutic of any one of claims 24-28, wherein the antibody is a pH sensitive binding antibody.

30. The targeted therapeutic of claim 24, wherein the antibody is a IgG2delta A and κ chain antibody.

31. The targeted therapeutic of claim 24, wherein the antibody fragment is a single chain scFv.

32. A nucleic acid encoding the targeted therapeutic of any one of claims 1-31.

33. A vector comprising the nucleic acid sequence of claim 32.

34. A host cell comprising the vector of claim 33.

35. The host cell of claim 34, wherein the host cell is selected from the group consisting of a bacterial, yeast, insect and mammalian cell.

36. The host cell of claim 35, wherein the host cell is a mammalian cell.

37. The host cell of claim 36, wherein the mammalian cell is a human cell.

38. The host cell of claim 36, wherein the mammalian cell is a CHO cell.

39. A method of producing a targeted therapeutic, the method comprising steps of:
a) culturing a host cell of any one of claims 34-38 under conditions suitable for expression of the targeted therapeutic by the host cell; and
b) harvesting the targeted therapeutic expressed by the host cell.

40. A pharmaceutical composition comprising the targeted therapeutic of any one of claims 1-31, and a pharmaceutical acceptable carrier.

41. A method of treating a lysosomal storage disease comprising administering to a subject in need of treatment the pharmaceutical composition of claim 40.


SEQUENCE LISTING (15 sequences)

SEQ ID NO: 1 - protein, 883 aa, Homo sapiens
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 925 aa, Homo sapiens
GHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 3 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 4 - protein, 720 aa, Homo sapiens
DEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 5 - protein, 743 aa, Homo sapiens
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 6 - protein, 15 aa, Artificial Sequence
  source: /note=“Description of Artificial Sequence Synthetic peptide”
GGGGGAAAAAGGGGG

SEQ ID NO: 7 - protein, 3 aa, Artificial Sequence
  source: /note=“Description of Artificial Sequence Synthetic peptide”
GAP

SEQ ID NO: 8 - protein, 6 aa, Artificial Sequence
  source: /note=“Description of Artificial Sequence Synthetic peptide”
GGGGGP

SEQ ID NO: 9 - protein, 39 aa, Artificial Sequence
  source: /note=“Description of Artificial Sequence Synthetic polypeptide”
GAPGGGGGAAAAAGGGGGGAPGGGGGAAAAAGGGGGGAP

SEQ ID NO: 10 - protein, 57 aa, Artificial Sequence
  source: /note=“Description of Artificial Sequence Synthetic polypeptide”
GAPGGGGGAAAAAGGGGGGAPGGGGGAAAAAGGGGGGAPGGGGGAAAAAGGGGGGAP

SEQ ID NO: 11 - protein, 1349 aa, Artificial Sequence
  source: /note=“Description of Artificial Sequence Synthetic polypeptide”
MEFGLSWLFLVAILKGVQCQVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTWVVVDVSHEDPEVQFNWYVDGVEVHNAKTKPREEQFNSTFCVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 12 - protein, 1146 aa, Artificial Sequence
  source: /note=“Description of Artificial Sequence Synthetic polypeptide”
MEFGLSWLFLVAILKGVQCQVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSGGGGSGGGGSGGGGSDIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRGGGAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 13 - protein, 1186 aa, Artificial Sequence
  source: /note=“Description of Artificial Sequence Synthetic polypeptide”
MEFGLSWLFLVAILKGVQCQVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTWVVVDVSHEDPEVQFNWYVDGVEVHNAKTKPREEQFNSTFCVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 14 - protein, 983 aa, Artificial Sequence
  source: /note=“Description of Artificial Sequence Synthetic polypeptide”
MEFGLSWLFLVAILKGVQCQVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSGGGGSGGGGSGGGGSDIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRGGGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 15 - protein, 15 aa, Artificial Sequence
  source: /note=“Description of Artificial Sequence Synthetic peptide”
GGGGSGGGGSGGGGS